(19) United States
(12) Patent Application Publication
(10) Pub. No.: US 2007/0155998 A1
(43) Pub. Date: Jul. 5, 2007

(54) FLUORENYLENE COMPOUND AND ORGANIC LIGHT-EMITTING DEVICE USING SAME

(75) Inventors: AKIHITO SAITOH, Yokohama-shi (JP); NAOKI YAMADA, Tokyo (JP); KEIJI OKINAKA, Kawasaki-shi (JP); KOICHI SUZUKI, Yokohama-shi (JP); AKIHIRO SENOO, Kawasaki-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/555,728

(22) Filed: Nov. 2, 2006

(30) Foreign Application Priority Data
Dec. 20, 2005 (JP) 2005-366184

(51) Int. Cl. H01L 51/54; C09K 11/06; C07C 13/567

(52) U.S. Cl. 585/26; 585/27; 428/690; 428/917; 313/504; 313/506; 257/E51.049; 257/E51.051

(57) ABSTRACT

A novel pyrenylfluorenylene compound is provided which is represented by the general formula (1):

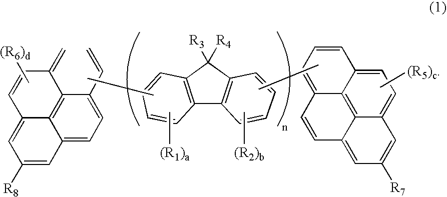

### BACKGROUND OF THE INVENTION

1. Field of the Invention

The present invention relates to a novel fluorenylene compound and an organic light-emitting device using the same.

2. Related Background Art

An organic light-emitting device has a structure in which a thin film comprising a fluorescent or phosphorescent organic compound is interposed between an anode and a cathode. By injecting electrons and holes (positive holes) from the electrodes into the device, excitons of the fluorescent or phosphorescent organic compound are generated, and light radiated when the excitons return to a ground state is utilized.

The recent progress of the organic light-emitting device is remarkable, and is characterized in that a highly responsive, thin, and lightweight light-emitting device that can be driven at a low applied voltage and provides a high luminance and a variety of emission wavelengths can be made, which suggests the applicability to a wide variety of uses.

However, at present, an optical output of a higher luminance or light emission of a higher conversion efficiency is still required. Further, there still remain a large number of problems in terms of durability such as a time-dependent change due to long-term use and deterioration due to an atmospheric gas containing oxygen or to moisture. Moreover, when application to a full-color display or the like is attempted, emission of blue, green and red lights with high color purities is necessary, but these problems have not satisfactorily been solved yet.

Japanese Patent Application Laid-Open No. 2002-324678 discloses that an organic light-emitting device using a material which has a pyrene-substituted benzene ring has good emission characteristics and durability, but the external quantum efficiency of the device is low, and the patent publication has no specific description about the durability life.

### SUMMARY OF THE INVENTION

It is, therefore, an object of the present invention to provide a novel pyrenylfluorenylene compound.

It is another object of the present invention to provide an organic light-emitting device which uses a substituted fluorenylene compound and has an optical output with a high efficiency and a high luminance.

It is still another object of the present invention to provide an organic light-emitting device which exhibits remarkably good durability.

It is yet another object of the present invention to provide an organic light-emitting device which can easily be produced at a relatively low cost.

The present inventors have conducted extensive studies in order to solve the above-mentioned problems and accomplished the present invention.

Namely, the fluorenylene compound according to the present invention is represented by the following general formula (1):

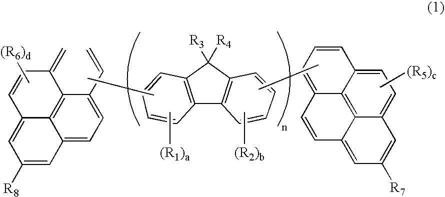

wherein the two pyrenyl groups are each independently bonded at position 1 or 4 thereof to the fluorenylene group; R₁ and R₂ each represent a substituted or unsubstituted alkyl group, a substituted or unsubstituted aralkyl group, a substituted or unsubstituted aryl group, a substituted or unsubstituted heterocyclic group, a substituted amino group, a cyano group, or a halogen atom, in which R₁'s and R₂'s which are bonded to different fluorene rings may be, respectively, the same or different from each other and R₁ and R₂ which are bonded to the same fluorene ring may be the same or different from each other, and when “a” and “b” are each an integer of 2 or more, R₁'s and R₂'s may be, respectively, the same or different from each other; R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group, in which R₃'s and R₄'s which are bonded to different fluorene rings may be, respectively, the same or different from each other and R₃ and R₄ which are bonded to the same fluorene ring may be the same or different from each other; R₅ represents a substituted or unsubstituted alkyl group, a substituted or unsubstituted aralkyl group, a substituted or unsubstituted alkoxy group, a substituted or unsubstituted aryl group, or a substituted or unsubstituted heterocyclic group, and when “c” is an integer of 2 or more, R₅'s may be the same or different from each other; R₆ represents a substituted or unsubstituted alkyl group having 1 or 2 carbons, a substituted or unsubstituted aralkyl group, a substituted or unsubstituted alkoxy group, a substituted or unsubstituted aryl group, or a substituted or unsubstituted heterocyclic group, and when “d” is an integer of 2 or more, R₆'s may be the same or different from each other; R₇ and R₈ each independently represent an iso-propyl group, a sec-butyl group, a tert-butyl group, a 1-adamantyl group, a 2-adamantyl group, an iso-amyl group, or a substituted silyl group; “a” and “b” each represent an integer from 0 to 3, in which “a's” and “b's” on different fluorene rings may be, respectively, the same or different from each other; “c” and “d” each represent an integer from 0 to 8, “c”+“d” being an integer from 1 to 16; and “n” represents an integer from 1 to 10; or by the following general formula (2):

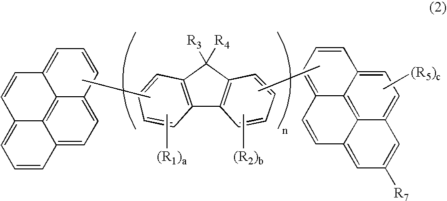

wherein the two pyrenyl groups are each independently bonded at position 1 or 4 thereof to the fluorenylene group; R₁ and R₂ each represent a substituted or unsubstituted alkyl group, a substituted or unsubstituted aralkyl group, a substituted or unsubstituted aryl group, a substituted or unsubstituted heterocyclic group, a substituted amino group, a cyano group, or a halogen atom, in which R₁'s and R₂'s which are bonded to different fluorene rings may be, respectively, the same or different from each other and R₁ and R₂ which are bonded to the same fluorene ring may be the same or different from each other, and when “a” and “b” are each an integer of 2 or more, R₁'s and R₂'s may be, respectively, the same or different from each other; R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group, in which R₃'s and R₄'s which are bonded to different fluorene rings may be, respectively, the same or different from each other and R₃ and R₄ which are bonded to the same fluorene ring may be the same or different from each other; R₅ represents a substituted or unsubstituted alkyl group, a substituted or unsubstituted aralkyl group, a substituted or unsubstituted alkoxy group, a substituted or unsubstituted aryl group, or a substituted or unsubstituted heterocyclic group, and when “c” is an integer of 2 or more, R₅'s may be the same or different from each other; R₇ represents an iso-propyl group, a sec-butyl group, a tert-butyl group, a 1-adamantyl group, a 2-adamantyl group, an iso-amyl group, or a substituted Silyl group; “a” and “b” each represent an integer from 0 to 3, in which “a's” and “b's” on different fluorene rings may be, respectively, the same or different from each other; “c” represents an integer from 1 to 8; and “n” represents an integer from 1 to 10.

Further, the organic light-emitting device in accordance with the present invention comprises a pair of electrodes including an anode and a cathode at least one of which is transparent or translucent, and at least one layer comprising an organic compound provided between the pair of electrodes, wherein at least one layer of the at least one layer comprising the organic compound comprises at least one of the above-mentioned fluorenylene compounds.

When the fluorenylene compound represented by the general formula (1) or (2) is used, an organic light-emitting device can be produced by use of a vapor deposition or casting method, and a relatively low-cost, large-area organic light-emitting device can easily be produced.

Moreover, the organic light-emitting device in accordance with the present invention provides light emission with a high luminance at a low applied voltage and is also excellent in durability. Especially, the organic layer comprising the fluorenylene compound in accordance with the present invention is excellent as a light-emitting layer and is also excellent as an electron-transporting layer.

### BRIEF DESCRIPTION OF THE DRAWINGS

FIG. 1 is a schematic cross-sectional view showing an example of the organic light-emitting device in accordance with the present invention.

FIG. 2 is a schematic cross-sectional view showing another example of the organic light-emitting device in accordance with the present invention.

FIG. 3 is a schematic cross-sectional view showing still another example of the organic light-emitting device in accordance with the present invention.

FIG. 4 is a schematic cross-sectional view showing yet another example of the organic light-emitting device in accordance with the present invention.

FIG. 5 is a schematic cross-sectional view showing yet still another example of the organic light-emitting device in accordance with the present invention.

### DESCRIPTION OF THE EMBODIMENTS

The present invention will now be described in detail.

First, the fluorenylene compound according to the present invention will be described.

The fluorenylene compound according to the present invention can be used mainly as a material for an organic light-emitting device. When used for a light-emitting layer therein, the fluorenylene compound may be used singularly in the light-emitting layer, or alternatively, can be used as a dopant (guest) material or a host material, thereby providing a device having a high color purity, a high emission efficiency, and a long life.

The fluorenylene compound according to the present invention is characterized by incorporating into a fluorene unit two pyrene rings as highly efficient emission units, wherein at least one of the pyrene rings has at least two substituents. In the pyrene ring having at least two substituents, one of the substituents is a group selected from the group consisting of an iso-propyl group, a sec-butyl group, a tert-butyl group, a 1-adamantyl group, a 2-adamantyl group, an iso-amyl group and a substituted silyl group. The other substituent is a group selected from the group consisting of an alkyl group, an aralkyl group, an alkoxy group, an aryl group and a substituted or unsubstituted heterocyclic group; or a group selected from the group consisting of a substituted or unsubstituted alkyl group having 1 or 2 carbons, an aralkyl group, an alkoxy group, an aryl group and a substituted or unsubstituted heterocyclic group.

By such incorporation of a plurality of substituents into the pyrene ring, an increase in Tg (e.g., 198° C. for Exemplary Compound 3) and an improvement in film-forming properties can be achieved, whereby a stable amorphous film can be obtained. By the incorporation of substituents into the pyrene ring, intermolecular stacking is relieved, whereby the solubility improves, and the synthesis and purification processes become easier. Further, the electron-transporting pyrene unit, especially when used as a host material, provides a highly efficient light emission by further using a hole-transporting dopant and adjusting the carrier balance.

In addition to the above described consideration, it has been also considered that the fluorenylene compound in accordance with the present invention inhibits molecular vibration and thermal inactivation through a deuterium isotope effect.

The fluorenylene compound in accordance with the present invention has been made through molecular design on the basis of the above described consideration, and thus the present invention has been accomplished.

Examples of the substituted or unsubstituted alkyl group in the above described general formula (1) or (2) include, but are not limited to, methyl group, methyl-d1 group, methyl-d3 group, ethyl group, ethyl-d5 group, n-propyl group, n-butyl group, n-pentyl group, n-hexyl group, n-heptyl group, n-octyl group, n-decyl group, iso-propyl group, iso-propyl-d7 group, iso-butyl group, sec-butyl group, tert-butyl group, tert-butyl-d9 group, iso-pentyl group, neopentyl group, tert-octyl group, fluoromethyl group, difluoromethyl group, trifluoromethyl group, 2-fluoroethyl group, 2,2,2-trifluoroethyl group, perfluoroethyl group, 3-fluoropropyl group, perfluoropropyl group, 4-fluorobutyl group, perfluorobutyl group, 5-fluoropentyl group, 6-fluorohexyl group, chloromethyl group, trichloromethyl group, 2-chloroethyl group, 2,2,2-trichloroethyl group, 4-chlorobutyl group, 5-chloropentyl group, 6-chlorohexyl group, bromomethyl group, 2-bromoethyl group, iodomethyl group, 2-iodoethyl group, hydroxymethyl group, hydroxyethyl group, cyclopropyl group, cyclobutyl group, cyclopentyl group, cyclohexyl group, cyclopentylmethyl group, cyclohexylmethyl group, cyclohexylethyl group, 4-fluorocyclohexyl group, norbornyl group, and adamantyl group.

Examples of the substituted or unsubstituted aralkyl group include, but are not limited to, benzyl group, 2-phenylethyl group, 2-phenylisopropyl group, 1-naphthylmethyl group, 2-naphthylmethyl group, 2-(1-naphthyl)ethyl group, 2-(2-naphthyl)ethyl group, 9-anthrylmethyl group, 2-(9-anthryl)ethyl group, 2-fluorobenzyl group, 3-fluorobenzyl group, 4-fluorobenzyl group, 2-chlorobenzyl group, 3-chlorobenzyl group, 4-chlorobenzyl group, 2-bromobenzyl group, 3-bromobenzyl group, and 4-bromobenzyl group.

Examples of the substituted or unsubstituted aryl group include, but are not limited to, phenyl group, phenyl-d5 group, 4-methylphenyl group, 4-methoxyphenyl group, 4-ethylphenyl group, 4-fluorophenyl group, 4-trifluorophenyl group, 3,5-dimethylphenyl group, 2,6-diethylphenyl group, mesityl group, 4-tert-butylphenyl group, ditolylaminophenyl group, biphenyl group, terphenyl group, naphthyl group, naphthyl-d7 group, acenaphthylenyl group, anthryl group, anthryl-d9 group, phenanthryl group, phenanthryl-d9 group, pyrenyl group, pyrenyl-d9 group, acephenanthrylenyl group, aceanthrylenyl group, chrysenyl group, dibenzochrysenyl group, benzoanthryl group, benzoanthryl-d11 group, dibenzoanthryl group, naphthacenyl group, picenyl group, pentacenyl group, fluorenyl group, triphenylenyl group, perylenyl group, and perylenyl-d-11 group.

Examples of the substituted or unsubstituted heterocyclic group include, but are not limited to, pyrrolyl group, pyridyl group, pyridyl-d5 group, bipyridyl group, methylpyridyl group, pyrimidinyl group, pyrazinyl group, pyridazinyl group, terpyrrolyl group, thienyl group, thienyl-d4 group, terthienyl group, propylthienyl group, benzothienyl group, dibenzothienyl group, dibenzothienyl-d7 group, furyl group, furyl-d4 group, benzofuryl group, isobenzofuryl group, dibenzofuryl group, dibenzofuryl-d7 group, quinolyl group, quinolyl-d6 group, isoquinolyl group, quinoxalinyl group, naphthyridinyl group, quinazolinyl group, phenanthridinyl group, indolizinyl group, phenazinyl group, carbazolyl group, oxazolyl group, oxadiazolyl group, thiazolyl group, thiadiazolyl group, acridinyl group, and phenazinyl group.

Examples of the substituted or unsubstituted amino group (—NR′R″) include, but are not limited to, those wherein R′ and R″ are each independently a hydrogen atom, a deuterium atom, the above described substituted or unsubstituted alkyl, aralkyl, aryl, and heterocyclic groups, alkyl, alkenyl, alkynyl, aralkyl, or amino groups as linked by the below-mentioned substituted or unsubstituted arylene or divalent heterocyclic group, substituted silyl group, ether group, thioether group, or carbonyl group; for example, amino group, N-methylamino group, N-ethylamino group, N,N-dimethylamino group, N,N-diethylamino group, N-methyl-N-ethylamino group, N-benzylamino group, N-methyl-N-benzylamino group, N,N-dibenzylamino group, anilino group, N,N-diphenylamino group, N-phenyl-N-tolylamino group, N,N-ditolylamino group, N-methyl-N-phenylamino group, N,N-dianisolylamino group, N-mesityl-N-phenylamino group, N,N-dimesitylamino group, N-phenyl-N-(4-tert-butylphenyl)amino group, and N-phenyl-N-(4-trifluoromethylphenyl)amino group.

Examples of the substituted or unsubstituted alkoxy group include, but are not limited to, an alkyloxy group or aralkyloxy group which has the above described substituted or unsubstituted alkyl group or aralkyl group; an aryloxy group having the above described substituted or unsubstituted aryl group or heterocyclic group; for example, methoxy group, ethoxy group, propoxy group, 2-ethyl-octyloxy group, phenoxy group, 4-tert-butylphenoxy group, benzyloxy group, and thienyloxy group.

Examples of the substituents which the above described substituents may further posses include, but are not limited to, a deuterium atom; alkyl or aralkyl groups such as methyl group, ethyl group, n-propyl group, n-butyl group, n-pentyl group, n-hexyl group, n-heptyl group, n-octyl group, n-decyl group, iso-propyl group, iso-butyl group, sec-butyl group, tert-butyl group, iso-pentyl group, neopentyl group, tert-octyl group, benzyl group, and 2-phenylethyl group; alkoxy groups such as methoxy group, ethoxy group, propoxy group, 2-ethyl-octyloxy group, phenoxy group, 4-tert-butylphenoxy group, and benzyloxy group; aryl groups such as phenyl group, 4-methylphenyl group, 4-ethylphenyl group, 3-chlorophenyl group, 3,5-dimethylphenyl group, triphenylamino group, biphenyl group, terphenyl group, naphthyl group, anthryl group, phenanthryl group, and pyrenyl group; heterocyclic groups such as pyridyl group, bipyridyl group, methylpyridyl group, thienyl group, terthienyl group, propylthienyl group, furyl group, quinolyl group, carbazolyl group, and N-ethylcarbazolyl group; a halogen group, a hydroxyl group, a cyano group, and a nitro group.

Incidentally, hydrogen atom(s) constituting the fluorenylene compound according to the present invention may be isotope(s) such as deuterium atom(s) or the like.

Preferable examples of the fluorenylene compound according to the present invention include those of the general formula (1) wherein R₇ and R₈ are both tert-butyl groups; i.e. the compounds represented by the following general formula (3).

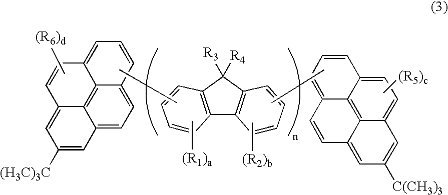

In addition, of the compounds represented by the general formula (3), more preferable compounds are those in which the pyrenyl group is bonded at position 1 thereof to position 2 or 7 of the fluorenylene group, “c” and “d” are both 1, and the pyrenyl groups are substituted at position 3 thereof with R₅ and R₆, respectively (i.e. compounds represented by the following general formula (4)).

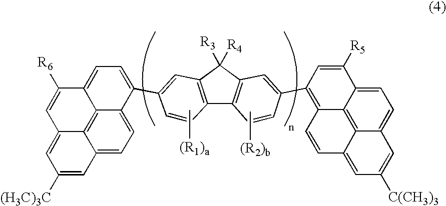

In addition, of the compounds of the general formula (4), more preferable compounds are those in which R₃, R₄, R₅, and R₆ are methyl groups, “a” and “b” are both 1, and “n” is from 1 to 3 (i.e. compounds represented by the following general formula (5)).

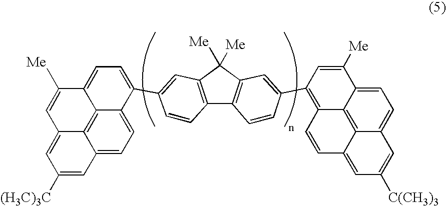

Next, typical examples of the fluorenylene compound according to the present invention will be given below. However, the fluorenylene compound according to the present invention is not limited to these compounds.

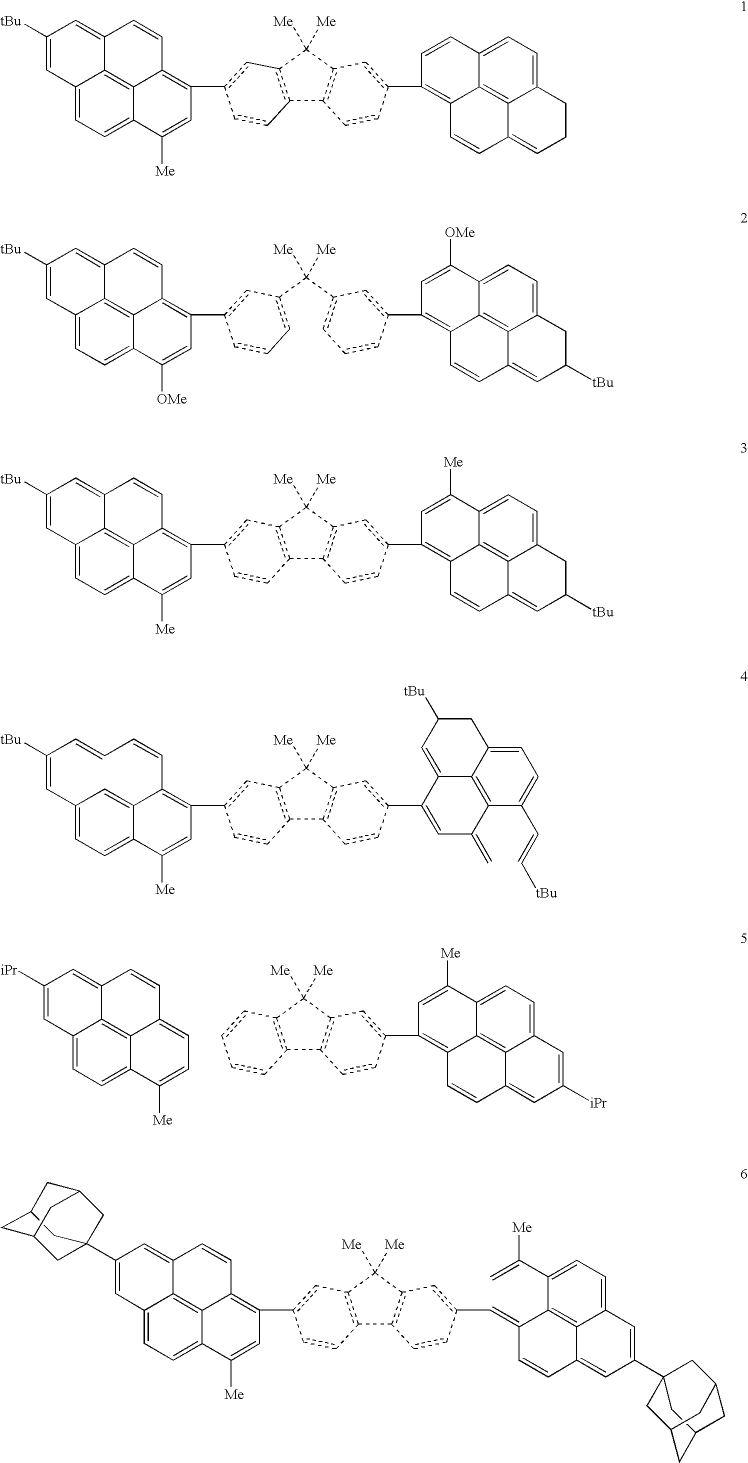

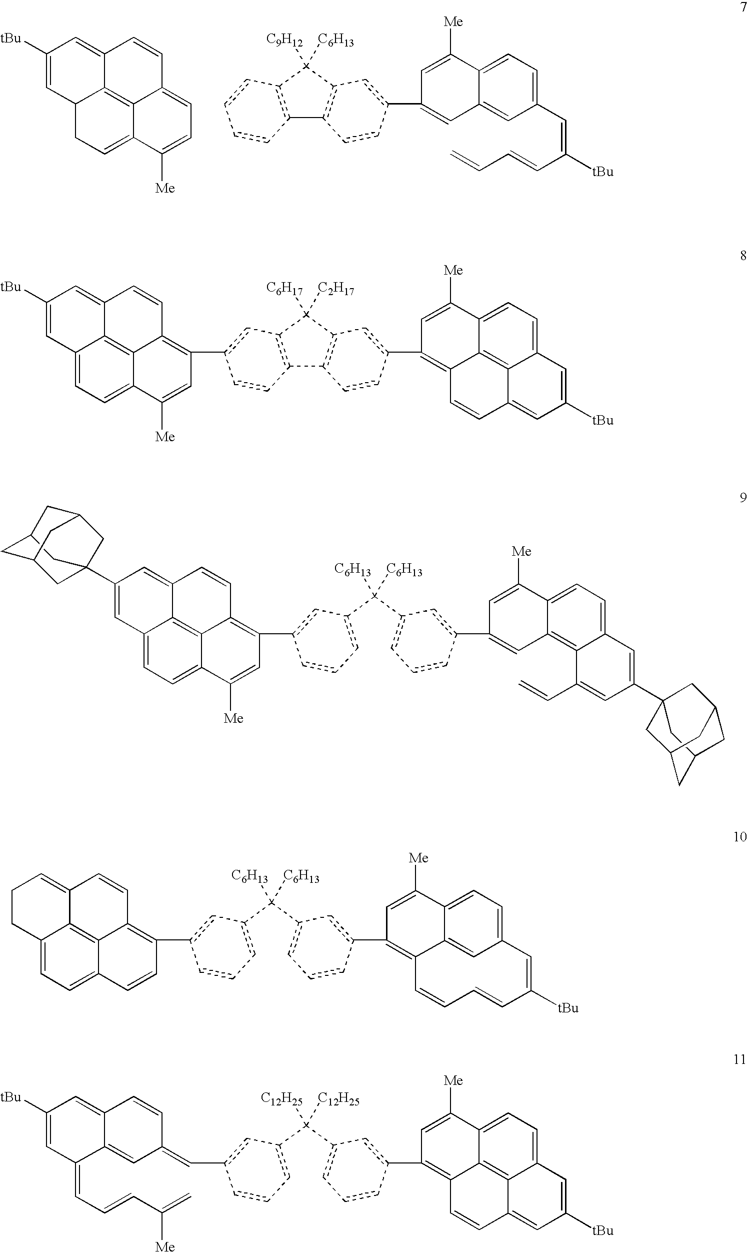

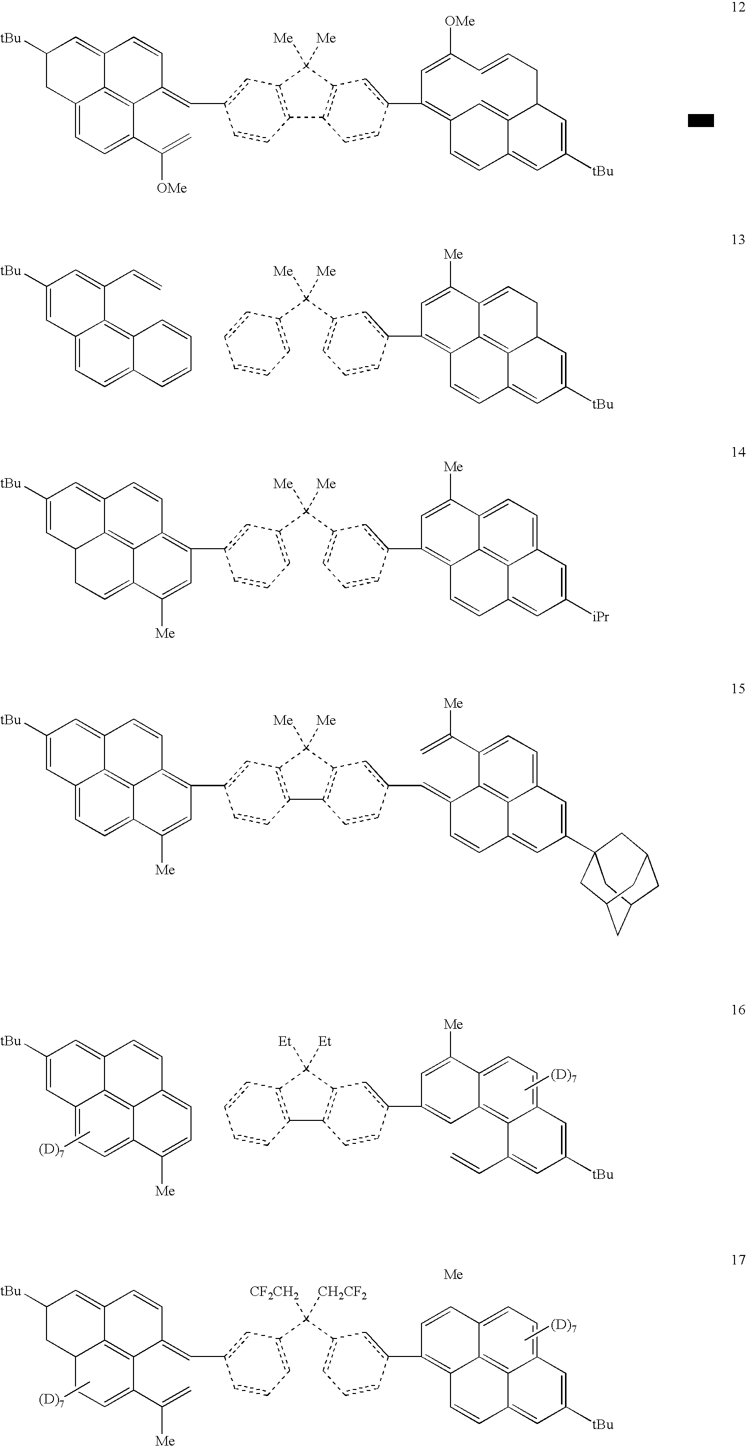

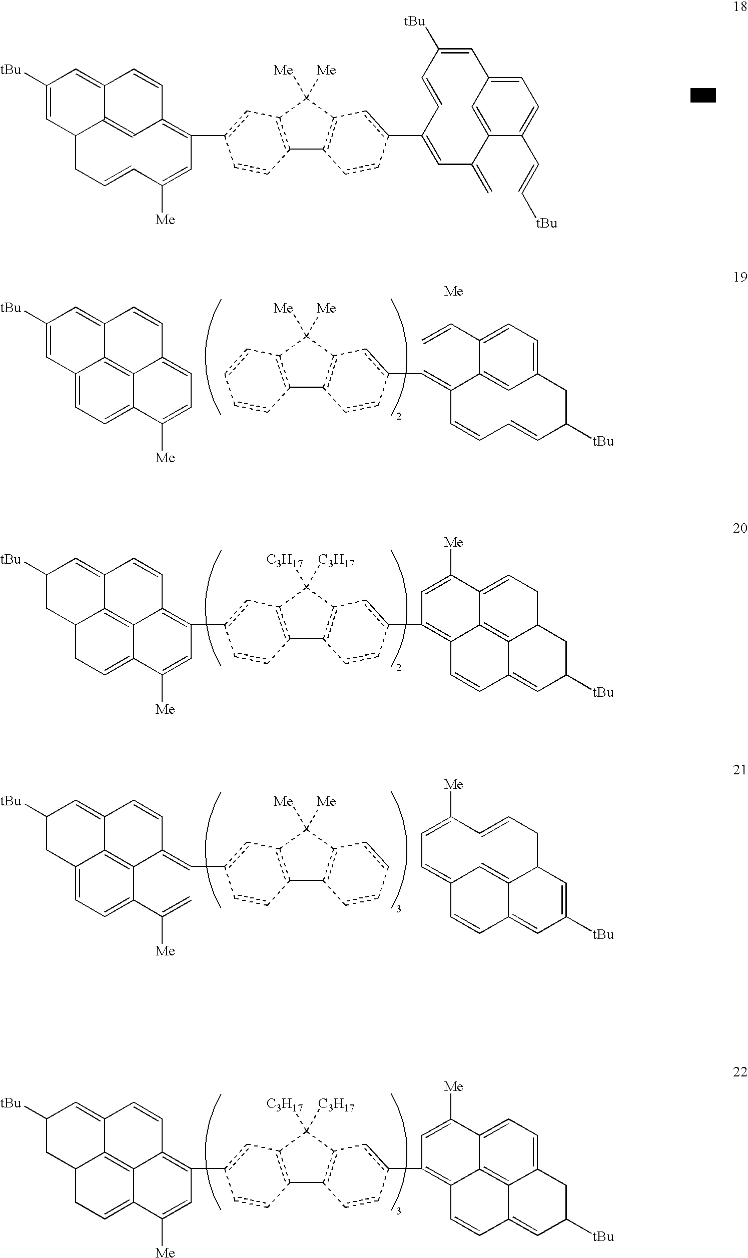

Next, the organic light-emitting device in accordance with the present invention will be described in detail below.

The organic light-emitting device in accordance with the present invention comprises a pair of electrodes including an anode and a cathode at least one of which is transparent or translucent, and at least one layer comprising an organic compound provided between the pair of electrodes. Further, at least one layer of the at least one layer comprising the organic compound, preferably a light-emitting layer, comprises at least one of the above-mentioned fluorenylene compounds.

In the organic light-emitting device in accordance with the present invention, it is preferred that the layer comprising at least one of the fluorenylene compounds in accordance with the present invention is a light-emitting layer. In this case, the light-emitting layer may be comprised only of the fluorenylene compound of the present invention, or may contain the fluorenylene compound of the present invention as a dopant (guest) material or host material.

When the fluorenylene compound of the present invention is used as a host material, the dopant concentration with respect to the host material is 0.01 wt. % or more and 80 wt. % or less, preferably 1 wt. % or more and 50 wt. % or less. The dopant material may be contained entirely in a layer comprised of the host material, either uniformly or with degraded concentration distribution, or may be contained locally, i.e., only in a region of the host material layer while leaving another region which contains no dopant material.

Preferable examples of compounds which can be used in combination with the fluorenylene compound according to the present invention include, but are not limited to, the arylamine compounds represented by the following general formulae (6) to (11) and (13) to (15), and the acetylene compounds represented by the following general formula (12).

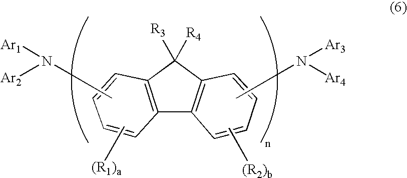

In the general formula (6), R₁ and R₂ each represent a substituted or unsubstituted alkyl group, a substituted or unsubstituted aralkyl group, a substituted or unsubstituted aryl group, a substituted or unsubstituted heterocyclic group, a substituted amino group, a cyano group, or a halogen atom, in which R₁'s and R₂'s which are bonded to different fluorene rings may be, respectively, the same or different from each other and R₁ and R₂ which are bonded to the same fluorene ring may be the same or different from each other, and when “a” and “b” are each an integer of 2 or more, R₁'s and R₂'s may be, respectively, the same or different from each other; R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group, in which R₃'s and R₄'s which are bonded to different fluorene rings may be, respectively, the same or different from each other and R₃ and R₄ which are bonded to the same fluorene ring may be the same or different from each other; Ar₁, Ar₂, Ar₃, and Ar₄ each represent a substituted or unsubstituted aromatic group, a substituted or unsubstituted heterocyclic group, a substituted or unsubstituted fused polycyclic aromatic group, or a substituted or unsubstituted fused heteropolycyclic group, in which Ar₁, Ar₂, Ar₃, and Ar₄ may be the same or different from one another, or Ar₁ and Ar₂, and Ar₃ and Ar₄ can be, respectively, joined to form a ring; “a” and “b” each represent an integer from 0 to 3, in which “a's” and “b's” for different fluorene rings may be, respectively, the same or different from each other; and “n” represents an integer from 1 to 10.

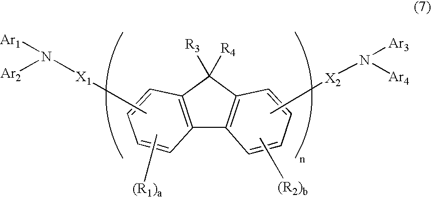

In the general formula (7), R₁ and R₂ each represent a substituted or unsubstituted alkyl group, a substituted or unsubstituted aralkyl group, a substituted or unsubstituted aryl group, a substituted or unsubstituted heterocyclic group, a substituted amino group, a cyano group, or a halogen atom, in which R₁'s and R₂'s which are bonded to different fluorene rings may be, respectively, the same or different from each other and R₁ and R₂ which are bonded to the same fluorene ring may be the same or different from each other, and when “a” and “b” are each an integer of 2 or more, R₁'s and R₂'s may be, respectively, the same or different from each other; R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group, in which R₃'s and R₄'s which are bonded to different fluorene rings may be, respectively, the same or different from each other and R₃ and R₄ which are bonded to the same fluorene ring may be the same or different from each other; Ar₁, Ar₂, Ar₃, and Ar₄ each represent a substituted or unsubstituted aromatic group, a substituted or unsubstituted heterocyclic group, a substituted or unsubstituted fused polycyclic aromatic group, or a substituted or unsubstituted fused heteropolycyclic group, in which Ar₁, Ar₂, Ar₃, and Ar₄ may be the same or different from one another, or Ar₁ and Ar₂, and Ar₃ and Ar₄ can be, respectively, joined to form a ring; X₁ and X₂ each represent a divalent, substituted or unsubstituted aromatic group or substituted or unsubstituted heterocyclic group, in which X₁ and X₂ may be the same or different from one another; “a” and “b” each represent an integer from 0 to 3, in which “a's” and “b's” for different fluorene rings may be, respectively, the same or different from each other; and “n” represents an integer from 1 to 10.

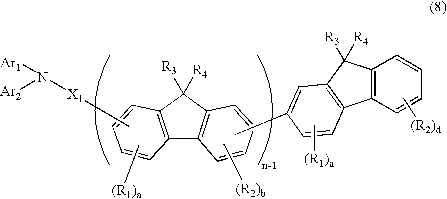

In the general formula (8), R₁ and R₂ each represent a substituted or unsubstituted alkyl group, a substituted or unsubstituted aralkyl group, a substituted or unsubstituted aryl group, a substituted or unsubstituted heterocyclic group, a substituted amino group, a cyano group, or a halogen atom, in which R₁'s and R₂'s which are bonded to different fluorene rings may be, respectively, the same or different from each other and R₁ and R₂ which are bonded to the same fluorene ring may be the same or different from each other, and when “a” and “b” are each an integer of 2 or more, R₁'s and R₂'s may be, respectively, the same or different from each other; R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group, in which R₃'s and R₄'s which are bonded to different fluorene rings may be, respectively, the same or different from each other and R₃ and R₄ which are bonded to the same fluorene ring may be the same or different from each other; Ar₁ and Ar₂ each represent a substituted or unsubstituted aromatic group, a substituted or unsubstituted heterocyclic group, a substituted or unsubstituted fused polycyclic aromatic group, or a substituted or unsubstituted fused heteropolycyclic group, in which Ar₁ and Ar₂ may be the same or different from one another or Ar₁ and Ar₂ can be joined to form a ring; and X₁ represents a divalent, substituted or unsubstituted aromatic group or substituted or unsubstituted heterocyclic group; “a” and “b” each represent an integer from 0 to 3, in which “a's” and “b's” for different fluorene rings may be, respectively, the same or different from each other; “d” represents an integer from 0 to 4; and “n” represents an integer from 1 to 10.

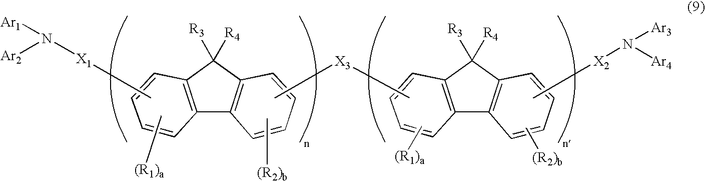

In the general formula (9), R₁ and R₂ each represent a substituted or unsubstituted alkyl group, a substituted or unsubstituted aralkyl group, a substituted or unsubstituted aryl group, a substituted or unsubstituted heterocyclic group, a substituted amino group, a cyano group, or a halogen atom, in which R₁'s and R₂'s which are bonded to different fluorene rings may be, respectively, the same or different from each other and R₁ and R₂ which are bonded to the same fluorene ring may be the same or different from each other, and when “a” and “b” are each an integer of 2 or more, R₁'s and R₂'s may be, respectively, the same or different from each other; R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group, in which R₃'s and R₄'s which are bonded to different fluorene rings may be, respectively, the same or different from each other and R₃ and R₄ which are bonded to the same fluorene ring may be the same or different from each other; Ar₁, Ar₂, Ar₃, and Ar₄ each represent a substituted or unsubstituted aromatic group, a substituted or unsubstituted heterocyclic group, a substituted or unsubstituted fused polycyclic aromatic group, or a substituted or unsubstituted fused heteropolycyclic group, in which Ar₁, Ar₂, Ar₃, and Ar₄ may be the same or different from one another, and Ar₁ and Ar₂, and Ar₃ and Ar₄ can be, respectively, joined to form a ring; X₁ and X₂ each represent a directly-bonded, divalent, substituted or unsubstituted aromatic group or substituted or unsubstituted heterocyclic group, which can be a group bonded through a linking group, in which X₁ and X₂ may be the same or different from one another; X₃ is a group selected from the group consisting of a substituted or unsubstituted alkylene group, alkenylene group, alkynylene group, aralkylene group, a divalent, substituted or unsubstituted aromatic group and substituted or unsubstituted heterocyclic group, and can be a group bonded through a linking group; “a” and “b” each represent an integer from 0 to 3, in which “a's” and “b's” for different fluorene rings may be, respectively, the same or different from each other; and “n” and “n′” each represent an integer from 1 to 10.

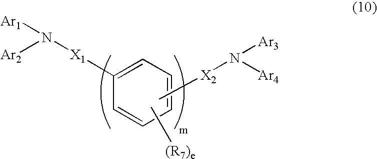

In the general formula (10), R₇ represents a substituted or unsubstituted alkyl group, a substituted or unsubstituted aralkyl group, a substituted or unsubstituted aryl group, a substituted or unsubstituted heterocyclic group, a cyano group, or a halogen atom, in which R₇'s bonded to different phenylene groups may be the same or different from each other, and when “e” is an integer of 2 or more, R₇'s bonded to the same phenylene group may be the same or different from each other; Ar₁, Ar₂, Ar₃, and Ar₄ each represent a substituted or unsubstituted aromatic group, a substituted or unsubstituted heterocyclic group, a substituted or unsubstituted fused polycyclic aromatic group, or a substituted or unsubstituted fused heteropolycyclic group, in which Ar₁, Ar₂, Ar₃, and Ar₄ may be the same or different from one another, or Ar₁ and Ar₂, and Ar₃ and Ar₄ can be, respectively, joined to form a ring; X₁ and X₂ each represent a direct bond, or a divalent, substituted or unsubstituted aromatic group or substituted or unsubstituted heterocyclic group, which can be a group bonded through a linking group, in which X₁ and X₂ may be the same or different from one another; “e” represents an integer from 0 to 4, in which “e's” for different phenylene groups may be the same or different from each other; and “m” represents an integer from 1 to 10.

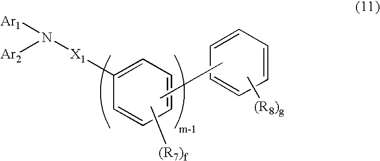

In the general formula (11), R₇ represents a substituted or unsubstituted alkyl group, a substituted or unsubstituted aralkyl group, a substituted or unsubstituted aryl group, a substituted or unsubstituted heterocyclic group, a cyano group, or a halogen atom, in which R₇'s bonded to different phenylene groups may be the same or different from each other, and when “f” is an integer of 2 or more, R₇'s bonded to the same phenylene group may be the same or different from each other; R₈ represents a substituted or unsubstituted alkyl group, a substituted or unsubstituted aralkyl group, a substituted or unsubstituted aryl group, a substituted or unsubstituted heterocyclic group, a cyano group, or a halogen atom, and when “g” is an integer of 2 or more, R₈'s may be the same or different from each other; Ar₁ and Ar₂ each represent a substituted or unsubstituted aromatic group, a substituted or unsubstituted heterocyclic group, a substituted or unsubstituted fused polycyclic aromatic group, or a substituted or unsubstituted fused heteropolycyclic group, in which Ar₁ and Ar₂ may be the same or different from one another or Ar₁ and Ar₂ can be joined to form a ring; X₁ represents a direct bond, or a divalent, substituted or unsubstituted aromatic group or substituted or unsubstituted heterocyclic group, which can be a group bonded through a linking group; “f” represents an integer from 0 to 4, in which “f's” for different phenylene groups may be the same or different from each other; g represents an integer from 0 to 5; and “m” represents an integer from 1 to 10.

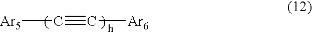

In the general formula (12), Ar₅ and Ar₆ each represent a substituted or unsubstituted aromatic group, a substituted or unsubstituted heterocyclic group, a substituted or unsubstituted fused polycyclic aromatic group, or a substituted or unsubstituted fused heteropolycyclic group, in which Ar₅ and Ar₆ may be the same or different from one another; and “h” represents an integer from 1 to 5.

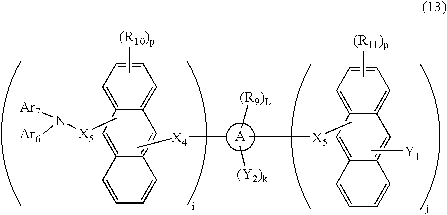

In the general formula (13), A represents a molecular unit comprising an aromatic ring, a fused polycyclic ring or a heterocyclic ring; Ar₇ and Ar₈ each represent a group selected from the group consisting of substituted or unsubstituted alkyl group, aralkyl group, aryl group, and heterocyclic group, in which Ar₇ and Ar₈ may be the same or different from each other, or Ar₇ and Ar₈ can be joined to form a ring, and Ar₇'s and Ar₈'s for different anthryl derivative groups may be, respectively, the same or different from each other; X₄ and X₅ are each a group selected from the group consisting of a direct bond, substituted or unsubstituted alkylene group, alkenylene group, alkynylene group, aralkylene group, and arylene group and a divalent heterocyclic group, which can be a group bonded through a linking group, and may be the same or different from one another, and X₄'s and X₅'s for different anthryl derivative groups may be, respectively, the same or different from each other; X₆ represents a direct bond, a divalent, substituted or unsubstituted aromatic group or substituted or unsubstituted heterocyclic group, which can be a group bonded through a linking group, and X₆'s for different anthryl derivative groups may be the same or different from each other; Y₁ is a group selected from the group consisting of a hydrogen atom, a halogen atom, substituted or unsubstituted alkyl group, alkenyl group, alkynyl group, aralkyl group, alkoxy group, sulfide group, aryl group, heterocyclic group, and boranyl group and a substituted silyl group, which may be a group bonded through a linking group, and Y₁'s for different anthryl derivative groups may be the same or different from each other; Y₂ is a group selected from the group consisting of substituted or unsubstituted aryl group and heterocyclic group, which may be a group bonded through a linking group, and when “k” is an integer of 2 or more, Y₂'s may be the same or different from each other; R₉ is a group selected from the group consisting of a hydrogen atom, a halogen atom, substituted or unsubstituted alkyl group and alkoxy group, and when “L” is an integer of 2 or more, R₉'s may be the same or different from each other; R₁₀ and R₁₁ are each a group selected from the group consisting of a halogen atom, substituted or unsubstituted alkyl group, aryl group, alkoxy group and amino group, and may be the same or different from each other, when “p” and “q” are each an integer of 2 or more, R₁₀'s and R₁₁'s may be, respectively, the same or different from each other, and R₁₀'s and R₁₁'s bonded to different anthracene rings may be, respectively, the same or different from each other; “i” is an integer from 0 to 6, and the relationship of j+k+L=6−i is satisfied with the proviso that the sum of i+j is an integer of 2 or more, and when “i” is zero at least one of Y₁'s on the anthryl groups contains a substituent other than a hydrogen atom and a halogen atom; and “p” is an integer from 0 to 8.

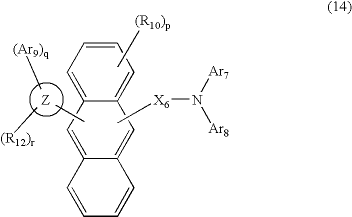

In the general formula (14), “Z” is a group selected from the group consisting of a direct bond, substituted or unsubstituted alkylene group, alkenylene group, alkynylene group, aralkylene group, and arylene group, and a divalent heterocyclic group, which can be a group bonded through a linking group; Ar₇ and Ar₈ each represent a group selected from the group consisting of substituted or unsubstituted alkyl group, aralkyl group, aryl group, and heterocyclic group, in which Ar₇ and Ar₈ may be the same or different from each other, or Ar₇ and Ar₈ can be joined to form a ring; Ar₉ represents a substituent comprising an aromatic fused polycyclic unit selected from the group consisting of substituted or unsubstituted naphthalene, phenanthrene, acenaphthylene, acephenanthrylene, aceanthrylene, triphenylene, chrysene, benzo[c]phenanthrene, naphthacene, dibenzo[a,c]anthracene, dibenzo[a,h]anthracene, dibenzo[b,def]chrysene, pyrene, picene, perylene, and pentacene, and a substituted or unsubstituted heterocyclic group, and when “q” is an integer of 2 or more, Ar₉'s may be the same or different from each other; X₆ represents a direct bond, a divalent, substituted or unsubstituted aromatic group or substituted or unsubstituted heterocyclic group, which can be a group bonded through a linking group, and X₆'s on different anthryl derivative groups may be the same or different from each other; R₁₀ is a group selected from the group consisting of a halogen atom, substituted or unsubstituted alkyl group, aryl group, alkoxy group, and amino group, and when “p” is an integer of 2 or more, R₁₀'s may be the same or different from each other; R₁₂ is a group selected from the group consisting of a halogen atom, substituted or unsubstituted alkyl group, aryl group excluding anthryl group, alkoxy group, and amino group, in which when “r” is an integer of 2 or more, R₁₂'s may be the same or different from each other; “p” represents an integer from 0 to 8; “q” represents an integer from 1 to 3; and “r” represents an integer from 0 to 4.

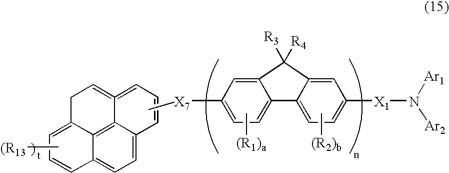

In the general formula (15), R₁ and R₂ each represent a substituted or unsubstituted alkyl group, a substituted or unsubstituted aralkyl group, a substituted or unsubstituted aryl group, a substituted or unsubstituted heterocyclic group, a substituted amino group, a cyano group, or a halogen atom, in which R₁'s and R₂'s which are bonded to different fluorene rings may be, respectively, the same or different from each other and R₁ and R₂ which are bonded to the same fluorene ring may be the same or different from each other, and when “a” and “b” are each an integer of 2 or more, R₁'s and R₂'s may be, respectively, the same or different from each other; R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group, in which R₃'s and R₄'s which are bonded to different fluorene rings may be, respectively, the same or different from each other and R₃ and R₄ which are bonded to the same fluorene ring may be the same or different from each other; R₁₃ is a group selected from the group consisting of a halogen atom, substituted or unsubstituted alkyl group, aryl group, alkoxy group, and amino group, and when “t” is an integer of 2 or more, R₁₃'s may be the same or different from each other; Ar₁ and Ar₂ each represent a substituted or unsubstituted aromatic group, a substituted or unsubstituted heterocyclic group, a substituted or unsubstituted fused polycyclic aromatic group, or a substituted or unsubstituted fused heteropolycyclic group, in which Ar₁ and Ar₂ may be the same or different from one another or Ar₁ and Ar₂ can be joined to form a ring; X₁ represents a direct bond, a divalent, substituted or unsubstituted aromatic group or substituted or unsubstituted heterocyclic group, which can be a group bonded through a linking group; X₇ is a group selected from the group consisting of substituted or unsubstituted alkylene group, alkenylene group, alkynylene group, aralkylene group, or a divalent, substituted or unsubstituted aromatic group or substituted or unsubstituted heterocyclic group, which may be a group bonded through a linking group; “a” and “b” each represent an integer from 0 to 3, in which “a's” and “b's” which for different fluorene rings may be, respectively, the same or different from each other; “t” represents an integer from 0 to 9; and “n” represents an integer from 1 to 10.

In the general formulae (6) to (15), examples of the substituted or unsubstituted alkyl group, aralkyl group, aryl group (aromatic group), heterocyclic group, amino group, and alkoxy group include, but are not limited to, those mentioned for the general formulae (1) and (2).

Examples of the substituted or unsubstituted alkenyl group include, but are not limited to, vinyl group, allyl group (2-propenyl group), 1-propenyl group, iso-propenyl group, 1-butenyl group, 2-butenyl group, 3-butenyl group and styryl group.

Examples of the substituted or unsubstituted alkynyl group include, but are not limited to, acetylenyl group, phenylacetylenyl group and 1-propynyl group.

Examples of the substituted or unsubstituted alkylene group include, but are not limited to, methylene group, ethylene group, propylene group, 2-methylpropylene group, fluoromethylene group, difluoromethylene group, bromomethylene group, and bromoethylene group.

Examples of the substituted or unsubstituted aralkylene group include, but are not limited to, benzylene group, 2-phenylethylene group, 2-phenylisopropylene group, 1-naphthylmethylene group, 2-naphthylmethylene group, 9-anthrylmethylene group, 2-fluorobenzylene group, 3-fluorobenzylene group, 4-fluorobenzylene group, 4-chlorobenzyl group, and 4-bromobenzylene group.

Examples of the substituted or unsubstituted alkenylene group include, but are not limited to, vinylene group, iso-propenylene group, styrylene group and 1,2-diphenylvinylene group.

Examples of the substituted or unsubstituted alkynylene group include, but are not limited to, acetylenylene group and a phenylacetylenylene group.

Examples of the substituted or unsubstituted divalent aromatic group include, but are not limited to, phenylene group, biphenylene group, tetrafluorophenylene group, dimethylphenylene group, naphthylene group, phenanthrylene group, pyrenylene group, tetracenylene group, pentacenylene group and perylenylene group.

Examples of the substituted or unsubstituted divalent heterocyclic group include, but are not limited to, furylene group, pyrrolylene group, pyridylene group, terpyridylene group, thienylene group, terthienylene group, oxazolylene group, thiazolylene group and carbazolylene group.

Examples of the linking group for bonding or joining of the above described substituents include, but are not limited to, the above described substituted or unsubstituted arylene, divalent heterocyclic, alkylene, alkenylene, alkynylene, and aralkylene groups; and substituted silyl group, ether group, thioether group, and carbonyl group.

Examples of the substituted or unsubstituted aralkylene group include, but are not limited to, benzylene group, 2-phenylethylene group, 2-phenylisopropylene group, 1-naphthylmethylene group, 2-naphthylmethylene group, 9-anthrylmethylene group, 2-fluorobenzylene group, 3-fluorobenzylene group, 4-fluorobenzylene group, 4-chlorobenzyl group, and 4-bromobenzylene group.

Examples of the substituted or unsubstituted alkenylene group include, but are not limited to, vinylene group, iso-propenylene group, styrylene group and 1,2-diphenylvinylene group.

Examples of the substituted or unsubstituted alkynylene group include, but are not limited to, acetylenylene group and a phenylacetylenylene group.

Examples of the substituted or unsubstituted arylene group (divalent aromatic group) include, but are not limited to, phenylene group, biphenylene group, tetrafluorophenylene group, dimethylphenylene group, naphthylene group, phenanthrylene group, pyrenylene group, tetracenylene group, pentacenylene group and perylenylene group.

Examples of the substituted or unsubstituted divalent heterocyclic group include, but are not limited to, furylene group, pyrrolylene group, pyridylene group, terpyridylene group, thienylene group, terthienylene group, oxazolylene group, thiazolylene group and carbazolylene group.

Examples of the substituted or unsubstituted sulfide group include, but are not limited to, an alkylsulfide group or aralkylsulfide group which has the above described substituted or unsubstituted alkyl group or aralkyl group; an arylsulfide group having the above described substituted or unsubstituted aryl group or heterocyclic group; for example, methyl sulfide group, ethylsulfide group, phenylsulfide group, and 4-methylphenylsulfide group.

Examples of the linking group for bonding or joining of the above described substituents include, but are not limited to, the above described substituted or unsubstituted arylene, divalent heterocyclic, alkylene, alkenylene, alkynylene, and aralkylene groups; and substituted silyl group, ether group, thioether group, and carbonyl group.

Examples of the substituents which the above-described substituents or linking group may further have include, but are not limited to, those mentioned for the general formulae (1) and (2).

Incidentally, hydrogen atom(s) constituting the compounds represented by the general formulae (6) to (15) may be isotope(s) such as deuterium atom(s) or the like.

Typical examples of the compounds represented by the general formula (6) will now be given. However, the present invention is not limited thereto.

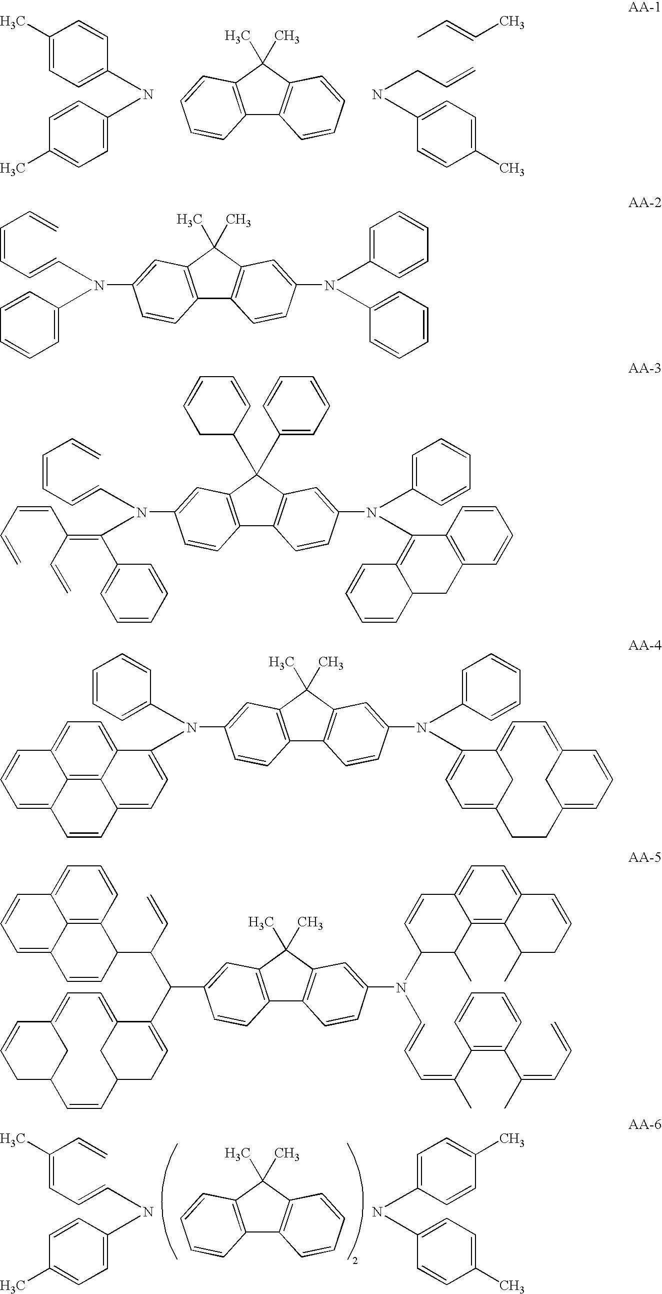

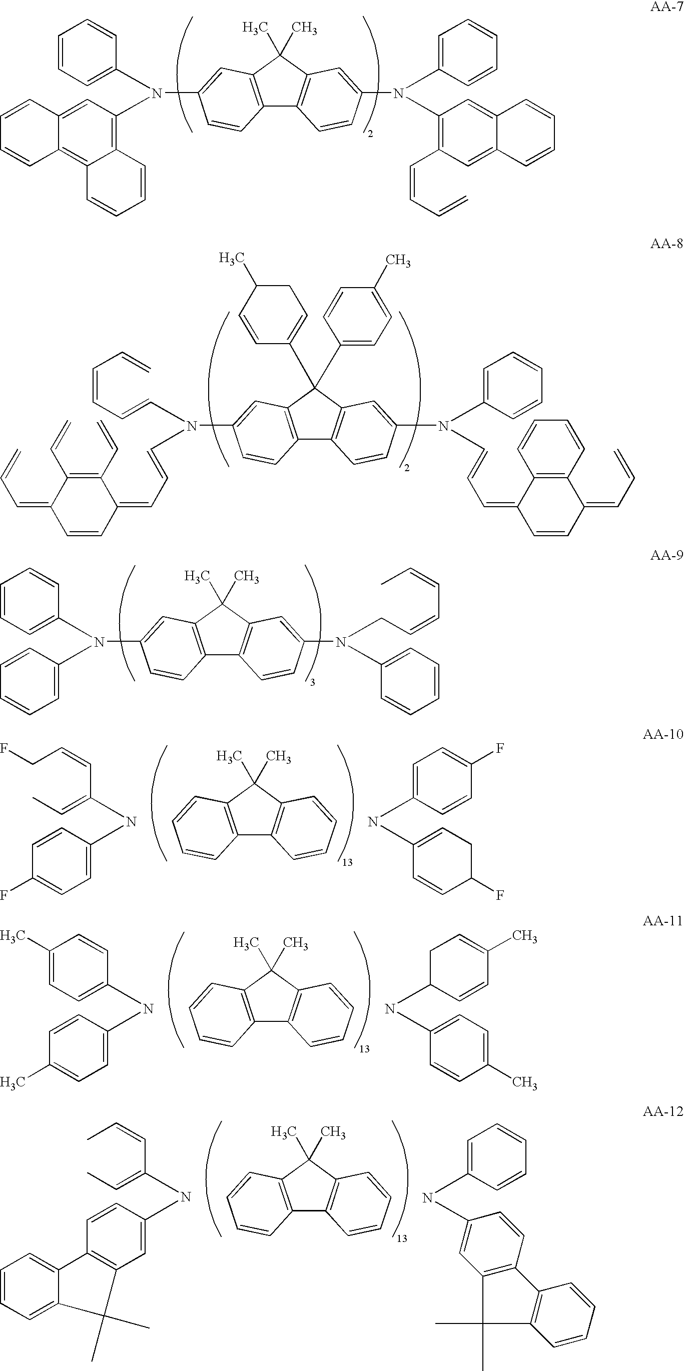

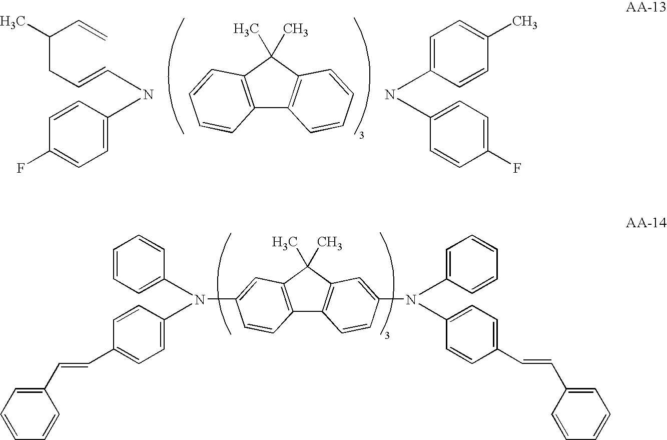

Next, typical examples of the compounds represented by the general formula (7) will be given. However, the present invention is not limited thereto.

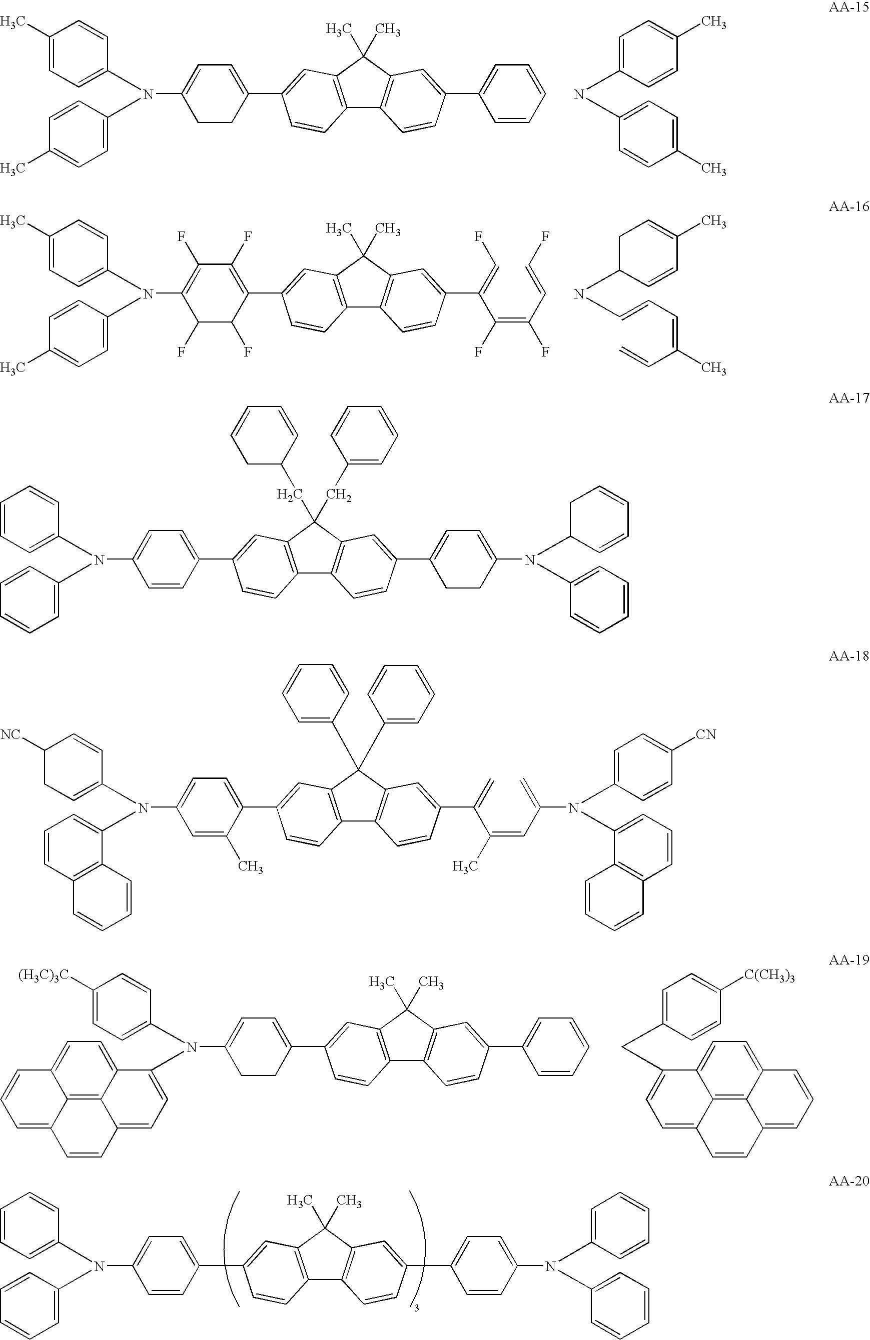

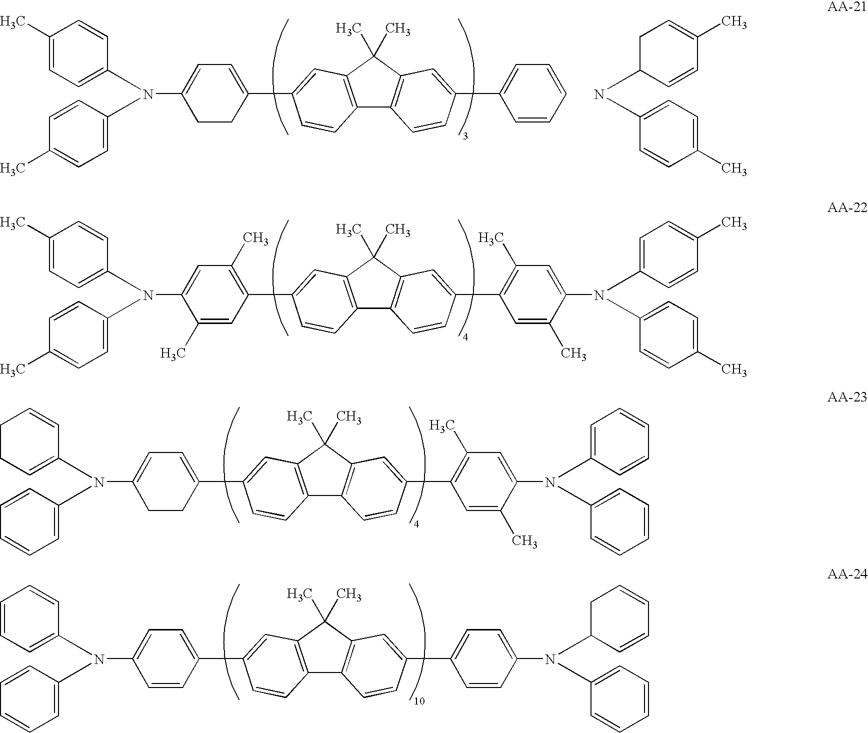

Next, typical examples of the compounds represented by the general formula (8) will be given. However, the present invention is not limited thereto.

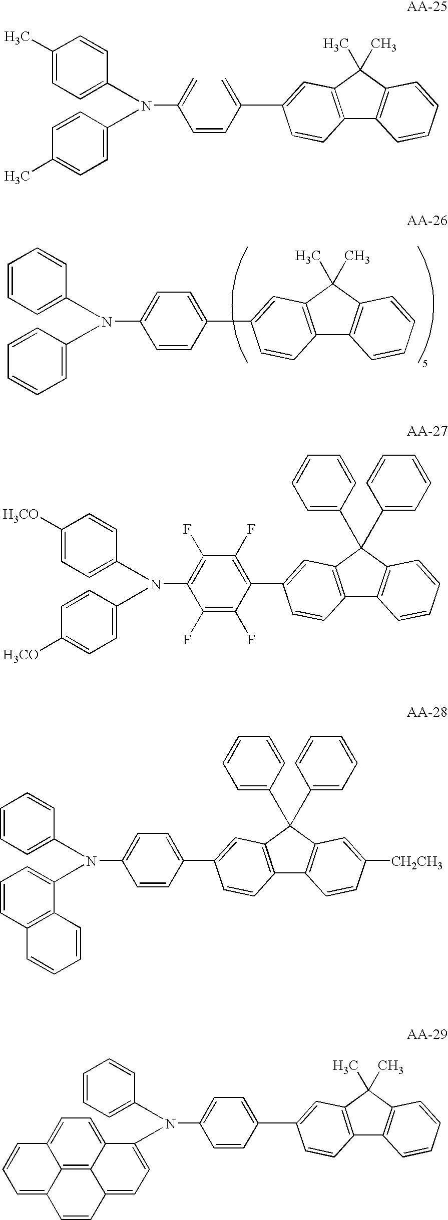

Next, typical examples of the compounds represented by the general formula (9) will be given. However, the present invention is not limited thereto.

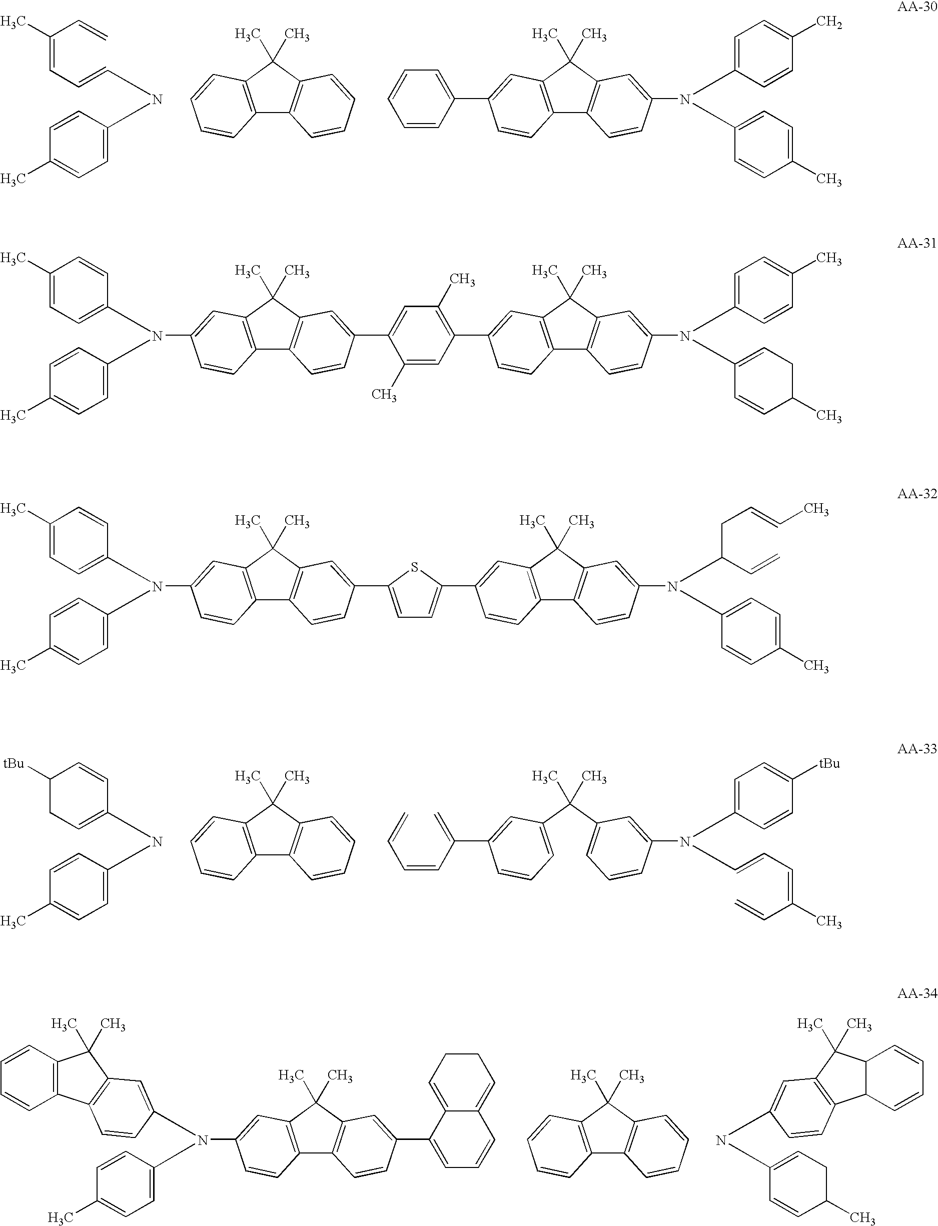

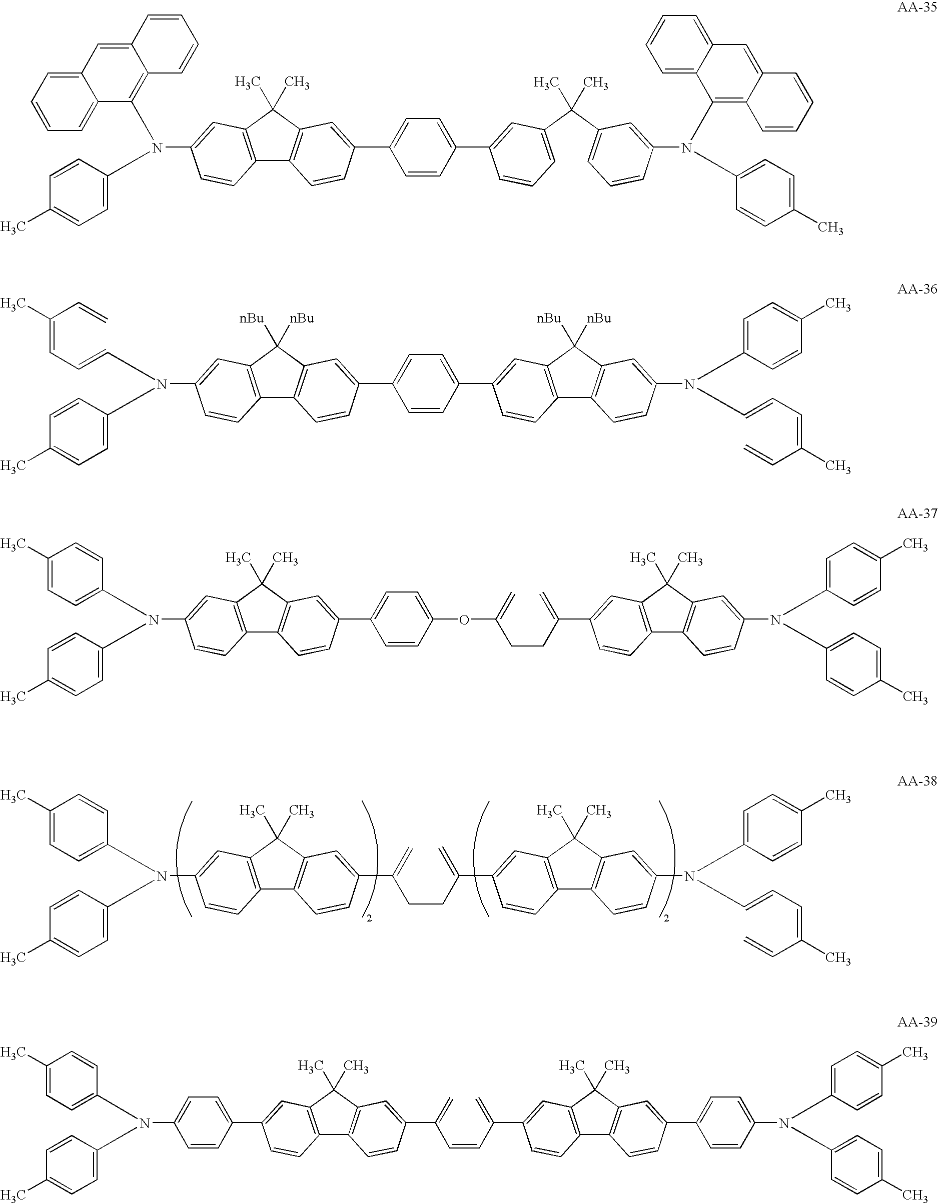

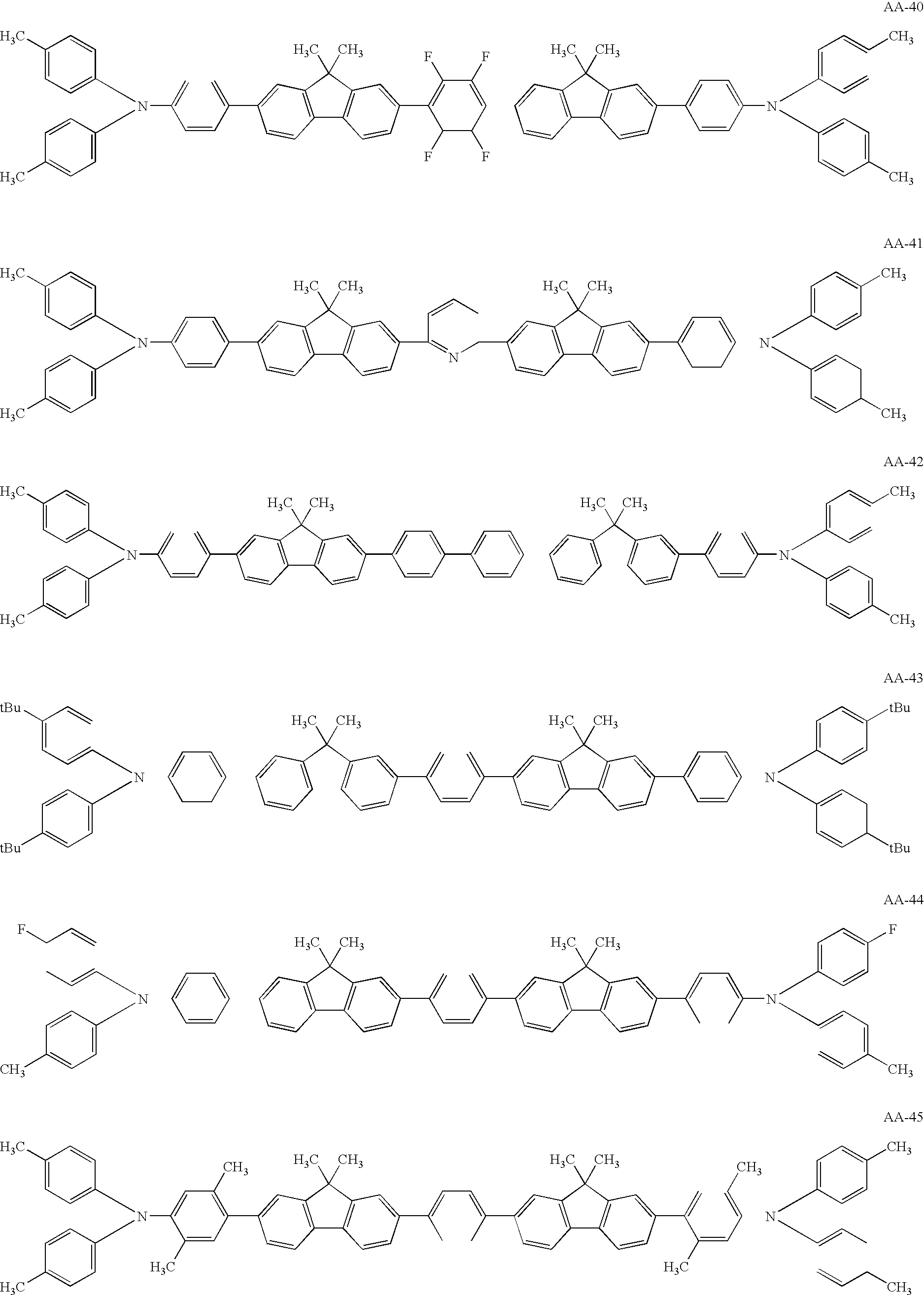

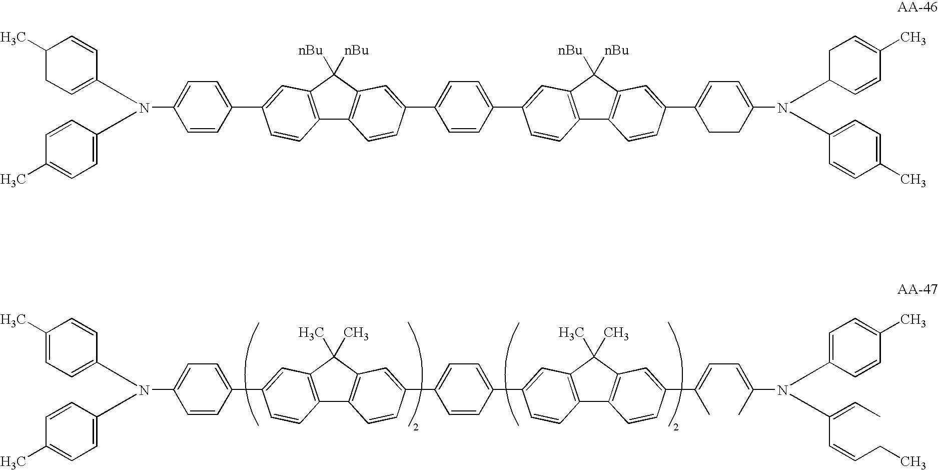

Next, typical examples of the compounds represented by the general formula (10) will be given. However, the present invention is not limited thereto.

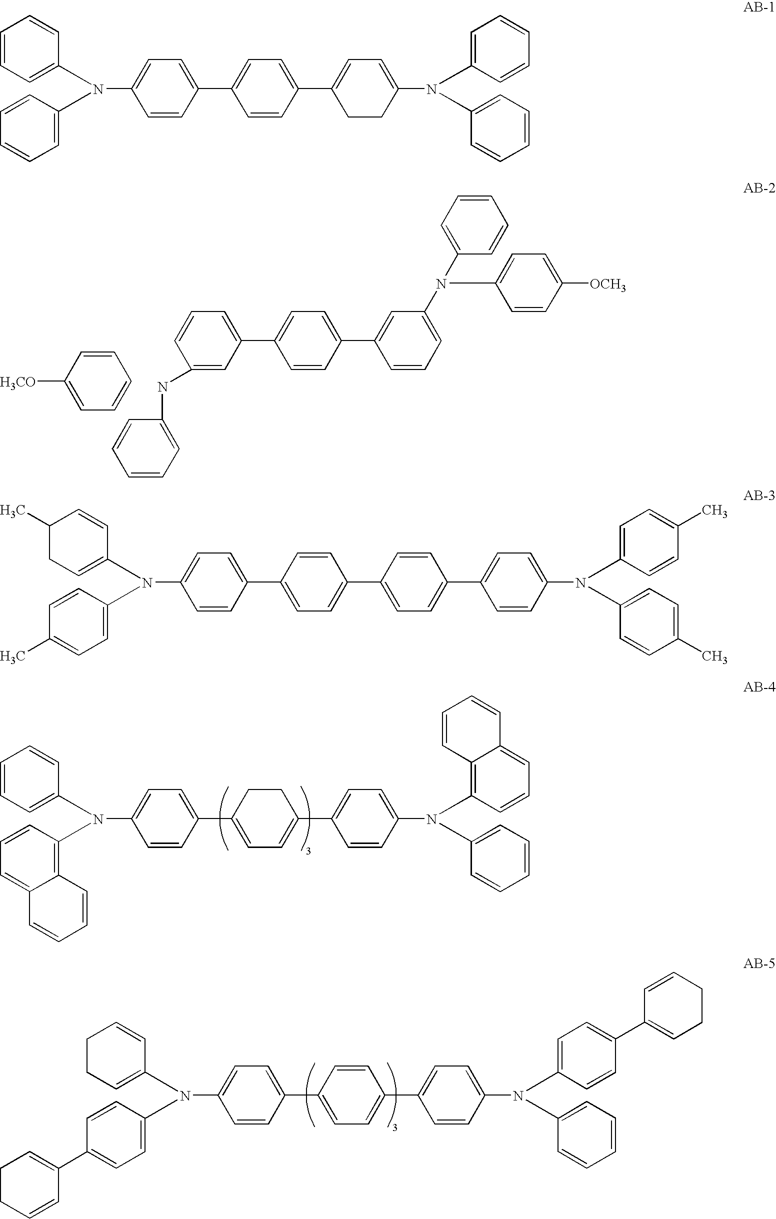

Next, typical examples of the compounds represented by the general formula (11) will be given. However, the present invention is not limited thereto.

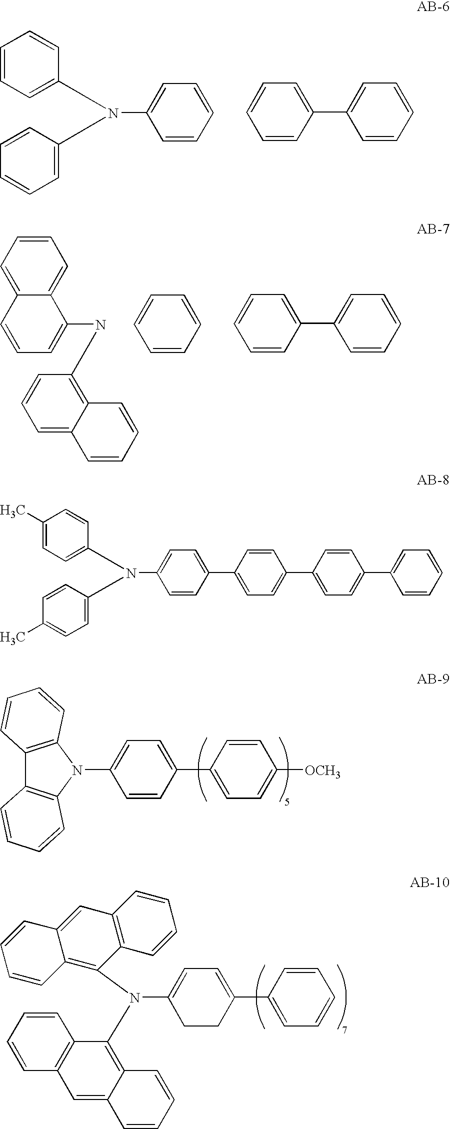

Next, typical examples of the compounds represented by the general formula (12) will be given. However, the present invention is not limited thereto.

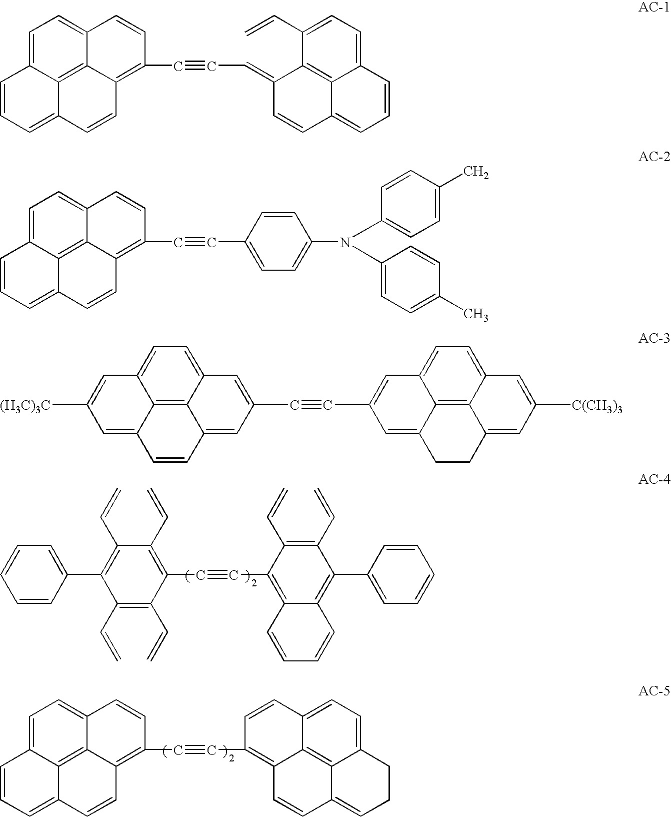

Next, typical examples of the compounds represented by the general formula (13) will be given. However, the present invention is not limited thereto.

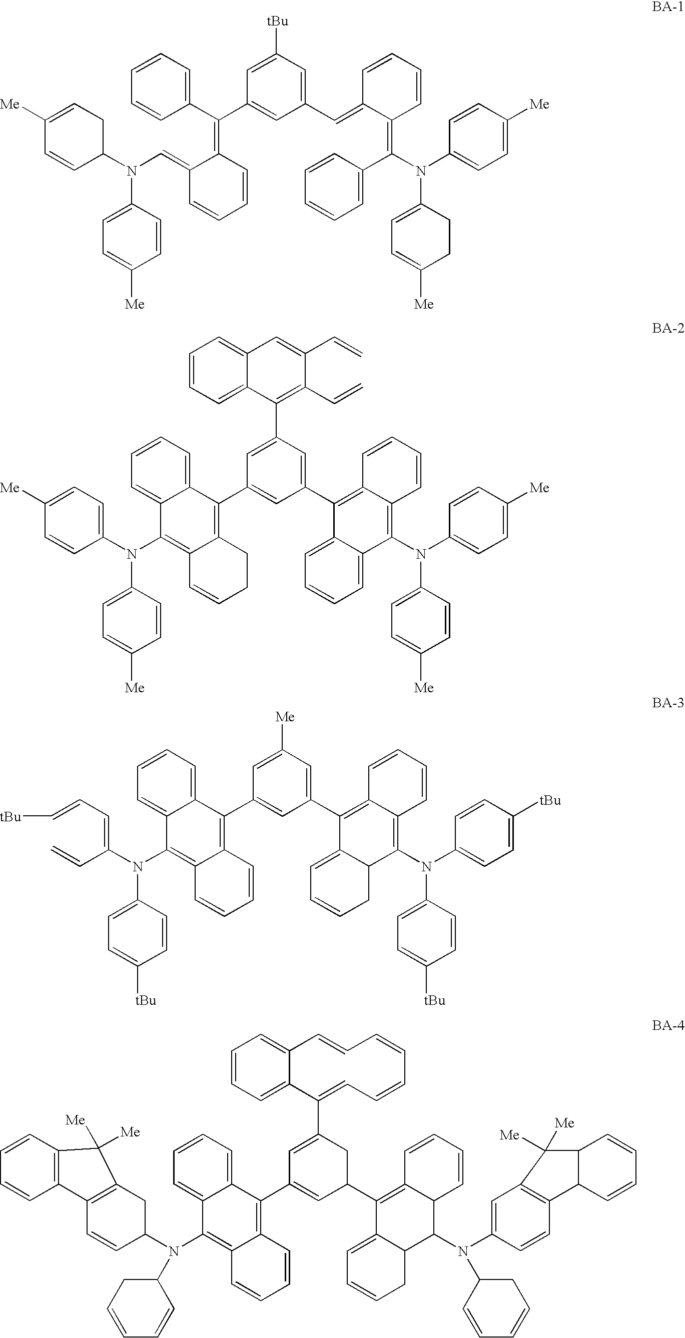

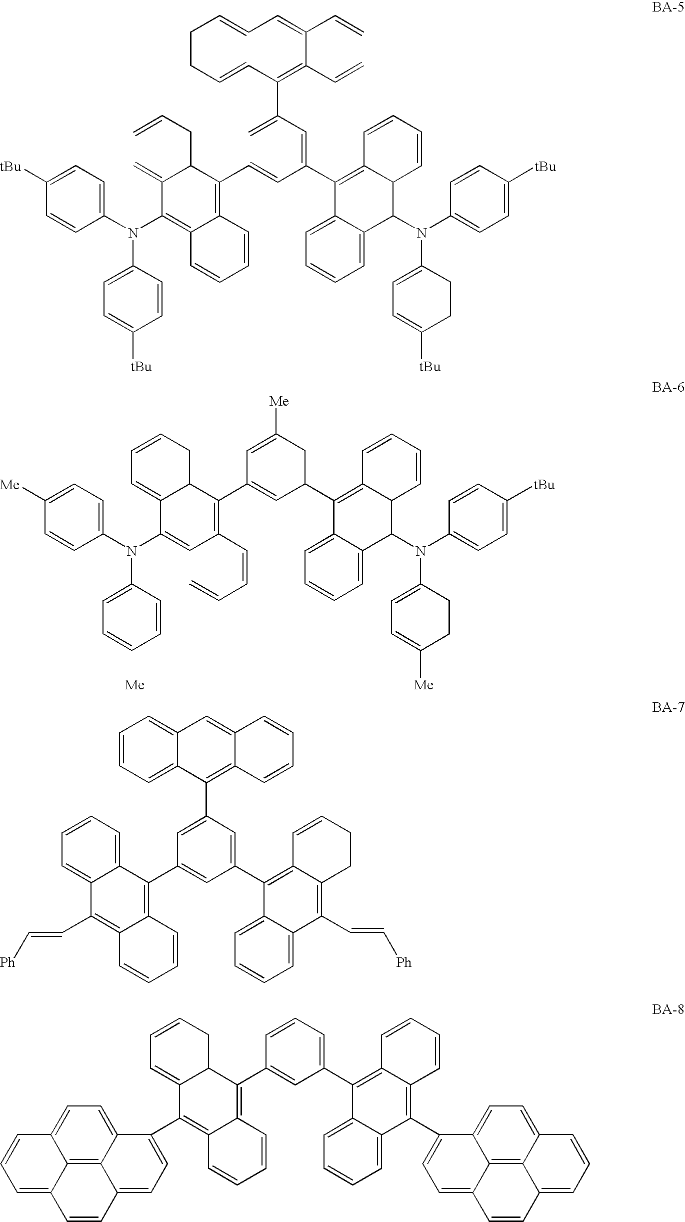

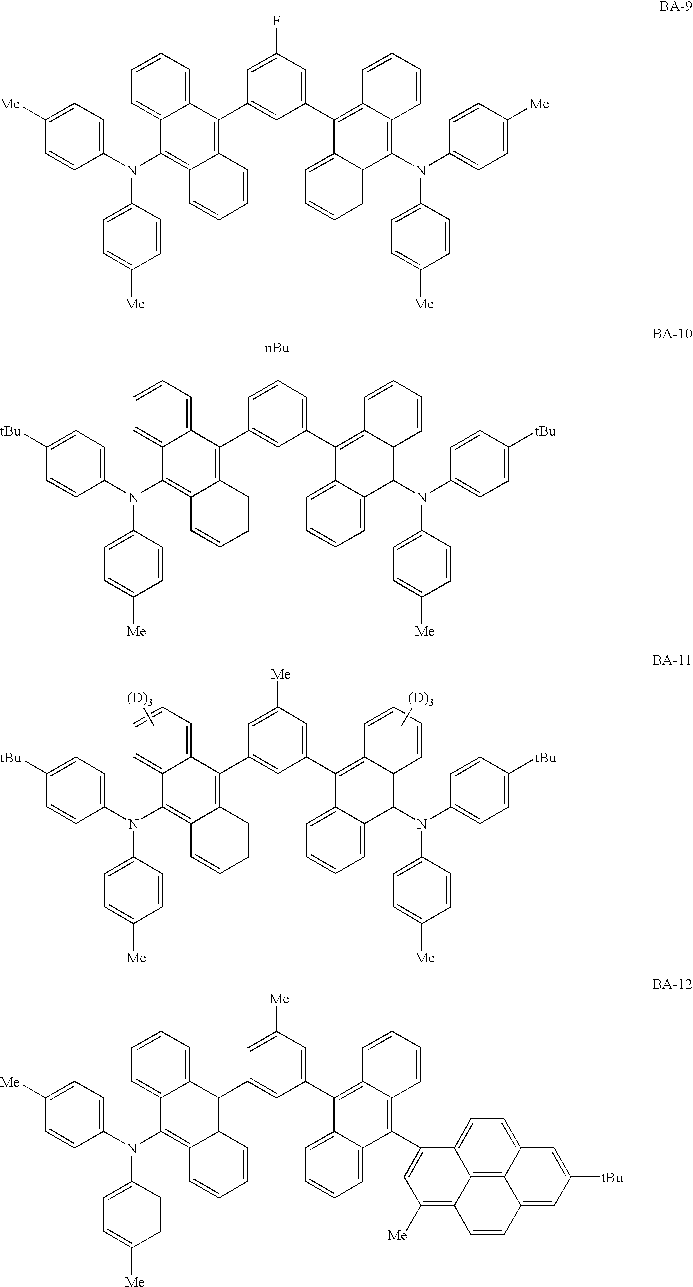

Next, typical examples of the compounds represented by the general formula (14) will be given. However, the present invention is not limited thereto.

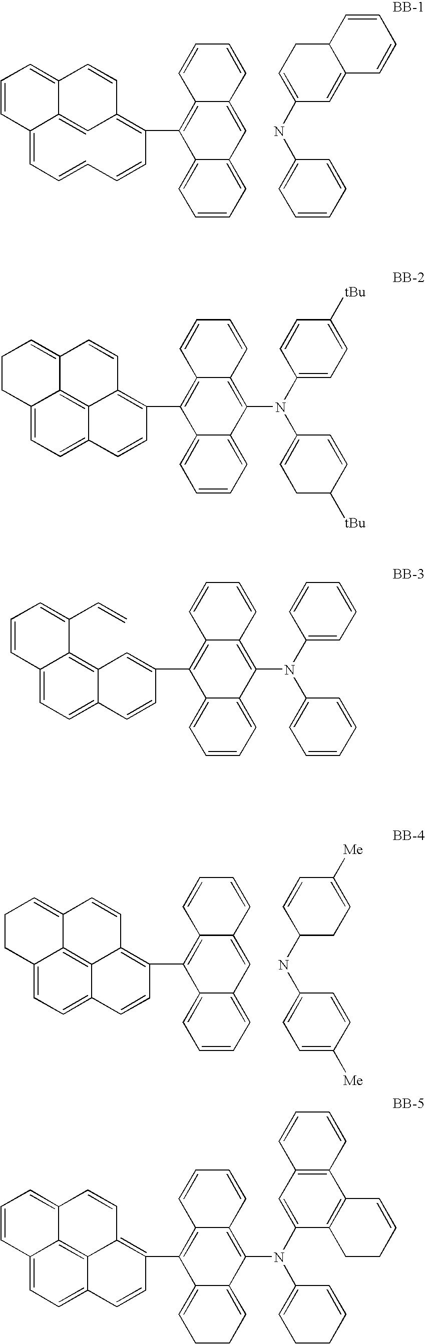

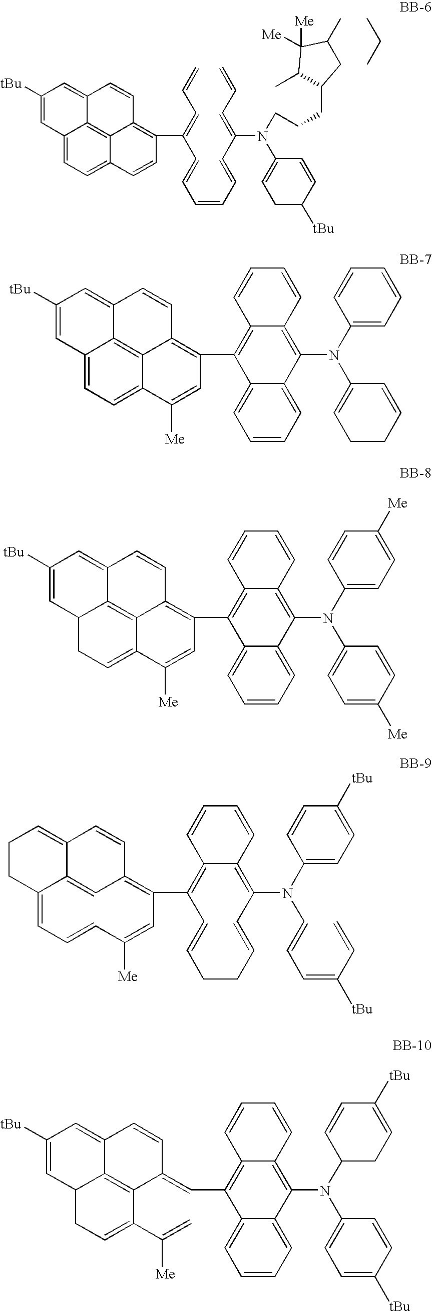

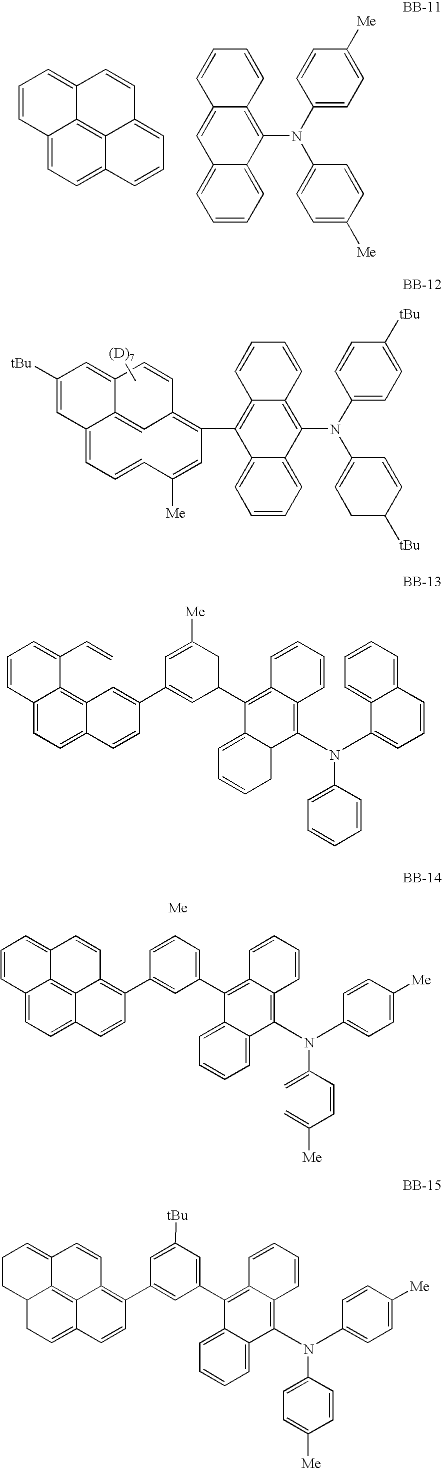

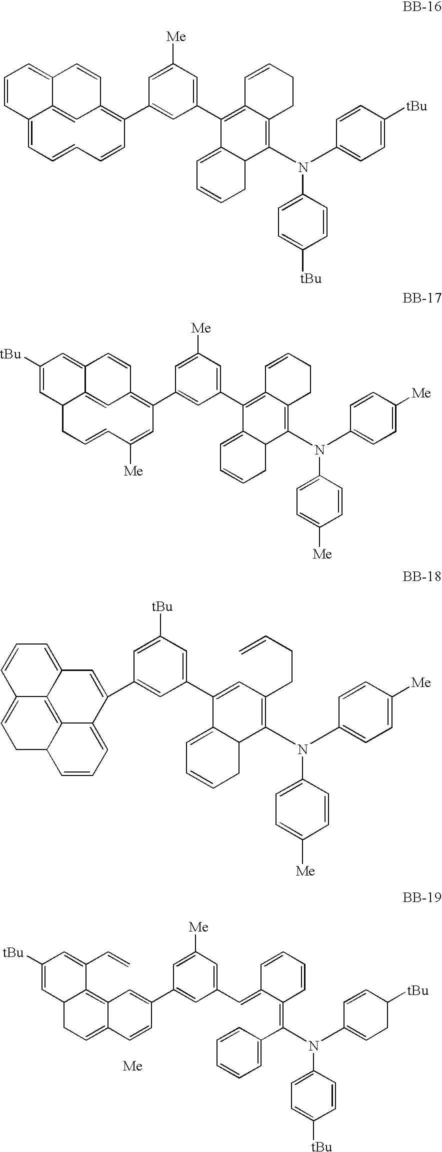

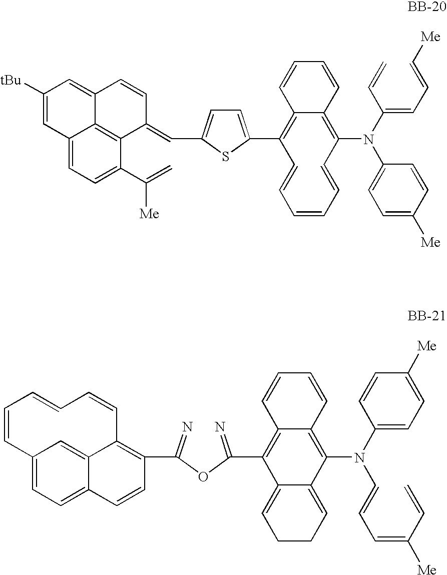

Next, typical examples of the compounds represented by the general formula (15) will be given. However, the present invention is not limited thereto.

TABLE 1

| Com-poundNo. | 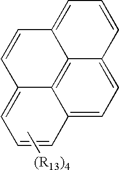 | X7 | 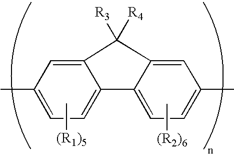 | X1 | Ar1 | Ar2 |
|---|---|---|---|---|---|---|
| C1 | 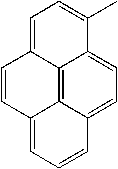 | directbond | 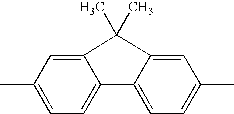 | directbond | 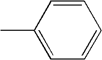 | 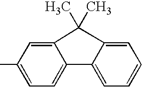 |
| C2 | 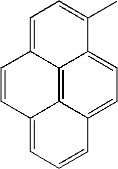 | directbond | 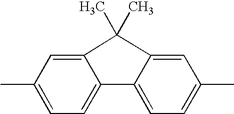 | directbond | 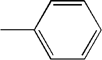 | 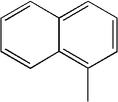 |
| C3 | 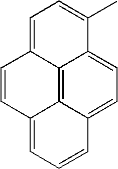 | directbond | 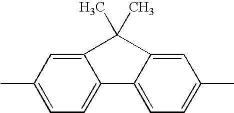 | directbond | 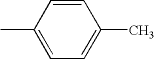 | 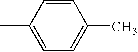 |
| C4 | 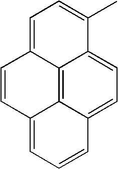 | directbond | 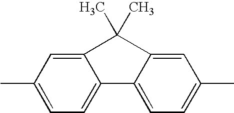 | directbond | 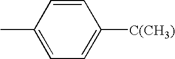 | 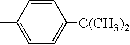 |
| C5 | 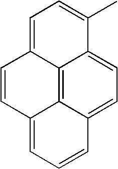 | directbond | 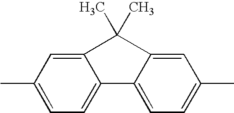 | directbond | 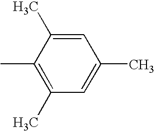 | 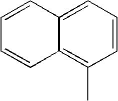 |
| C6 | 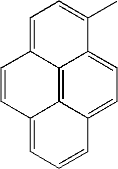 | directbond | 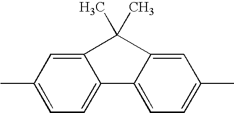 | directbond | 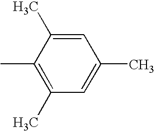 | 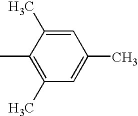 |
| C7 | 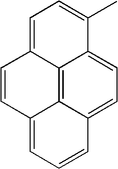 | directbond | 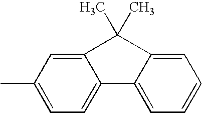 | directbond | 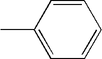 | 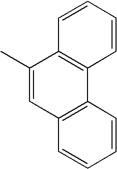 |
| C8 | 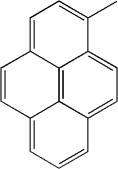 | directbond | 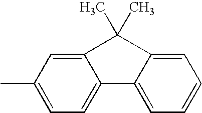 | directbond | 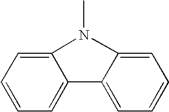 | |
| C9 | 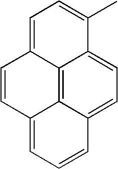 | directbond | 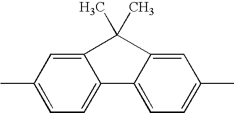 | 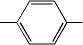 | 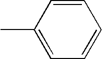 | 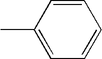 |

TABLE 2

| | | | | | | |
|---|---|---|---|---|---|---|
| C10 | 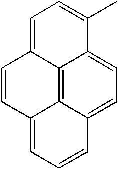 | directbond | 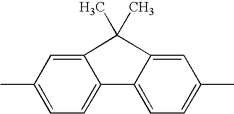 | 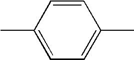 | 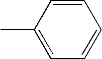 | 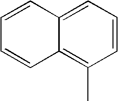 |
| C11 | 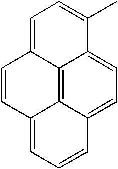 | directbond | 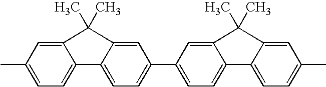 | 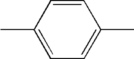 | 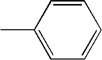 | 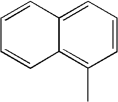 |
| C12 | 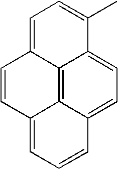 | directbond | 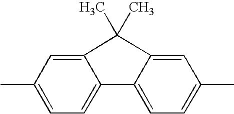 | 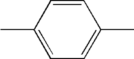 | 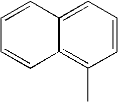 | 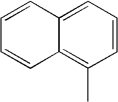 |
| C13 | 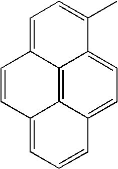 | directbond | 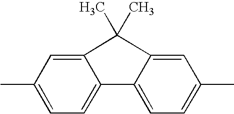 | 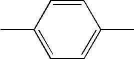 | 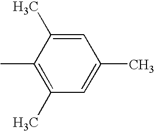 | 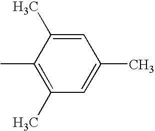 |
| C14 | 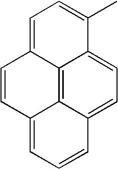 | directbond | 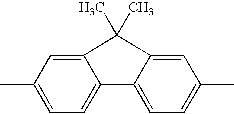 | 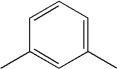 | 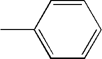 | 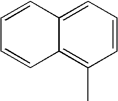 |
| C15 | 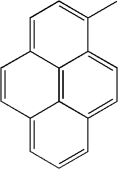 | directbond | 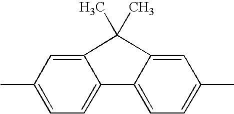 | 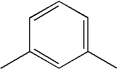 | 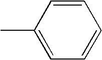 | 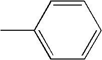 |
| C16 | 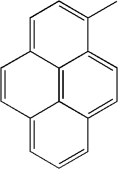 | directbond | 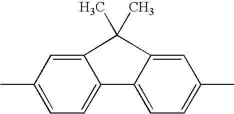 | 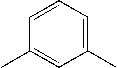 | 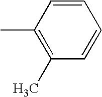 | 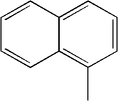 |
| C17 | 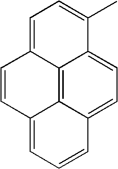 | directbond | 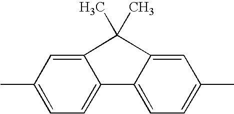 | 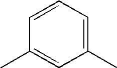 | 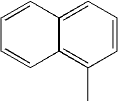 | 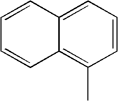 |
| C18 | 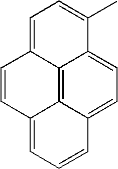 | directbond | 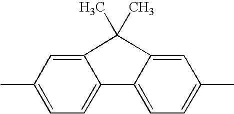 | 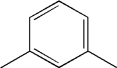 | 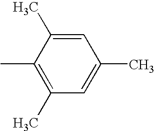 | 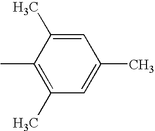 |
| C19 | 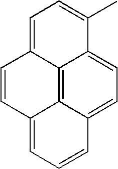 | directbond | 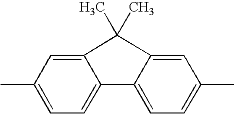 | 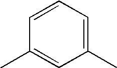 | 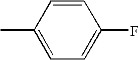 | 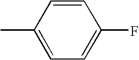 |

TABLE 3

| | | | | | | |
|---|---|---|---|---|---|---|
| C20 | 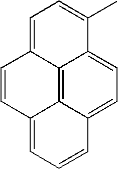 | directbond | 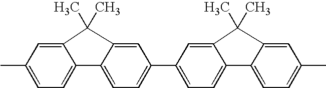 | 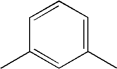 | 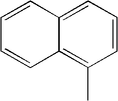 | 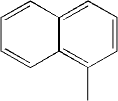 |
| C21 | 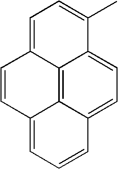 | directbond | 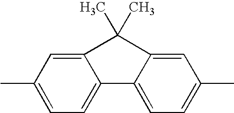 | 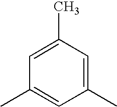 | 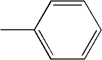 | 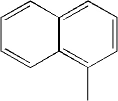 |
| C22 | 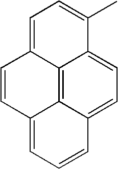 | directbond | 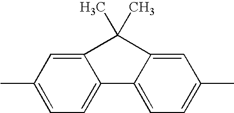 | 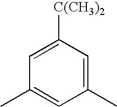 | 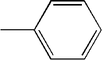 | 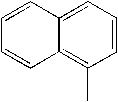 |
| C23 | 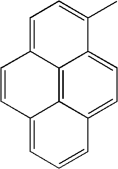 | directbond | 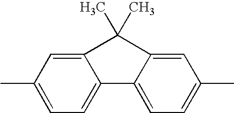 | 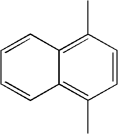 | 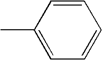 | 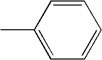 |
| C24 | 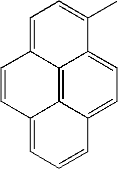 | directbond | 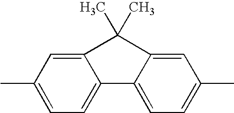 | 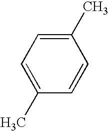 | 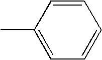 | 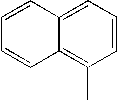 |
| C25 | 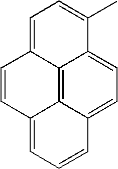 | directbond | 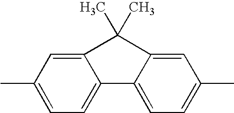 | 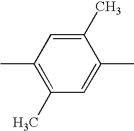 | 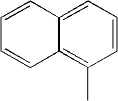 | 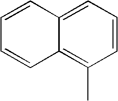 |
| C26 | 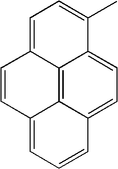 | 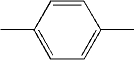 | 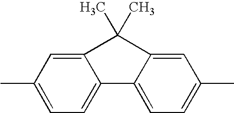 | 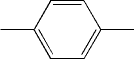 | 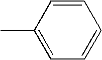 | 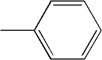 |
| C27 | 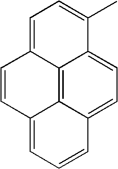 | 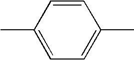 | 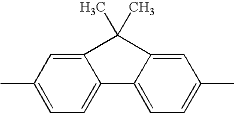 | 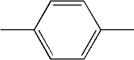 | 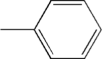 | 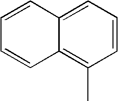 |
| C28 | 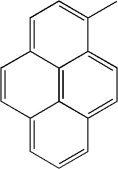 | 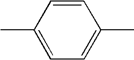 | 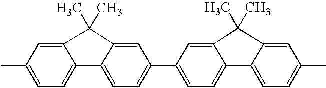 | 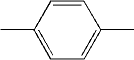 | 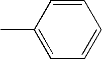 | 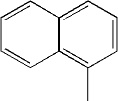 |
| C29 | 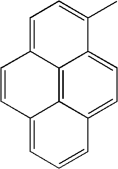 | 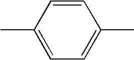 | 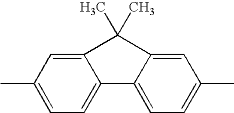 | 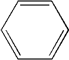 | 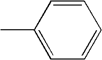 | 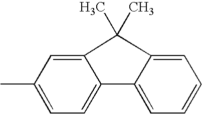 |

TABLE 4

| | | | | | | |
|---|---|---|---|---|---|---|
| C30 | 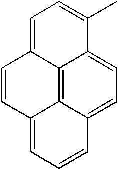 | 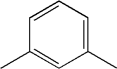 | 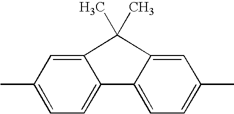 | 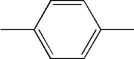 | 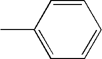 | 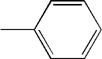 |
| C31 | 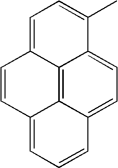 | 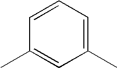 | 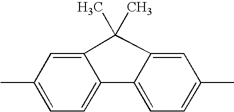 | 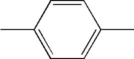 | 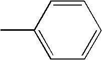 | 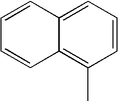 |
| C32 | 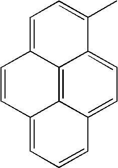 | 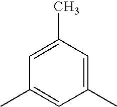 | 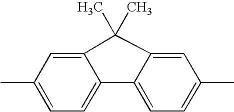 | 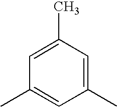 | 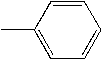 | 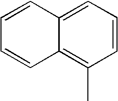 |
| C33 | 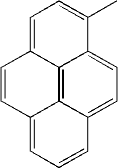 | 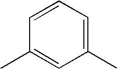 | 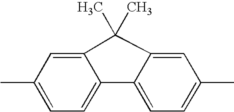 | 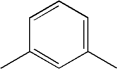 | 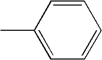 | 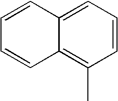 |
| C34 | 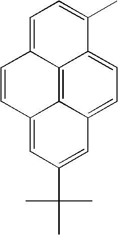 | directbond | 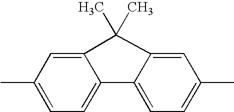 | directbond | 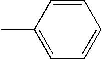 | 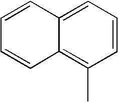 |
| C35 | 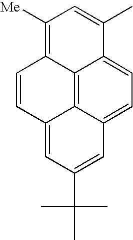 | directbond | 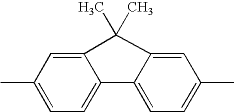 | directbond | 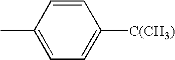 | 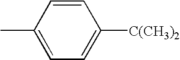 |
| C36 | 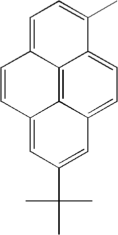 | directbond | 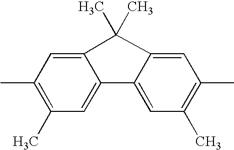 | 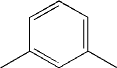 | 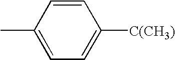 | 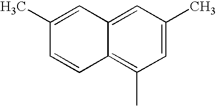 |
| C37 | 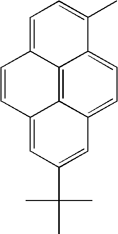 | directbond | 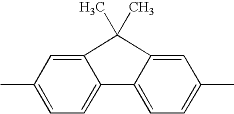 | 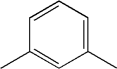 | 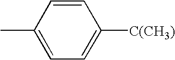 | 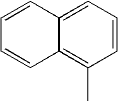 |

TABLE 5

| | | | | | | |
|---|---|---|---|---|---|---|
| C38 | 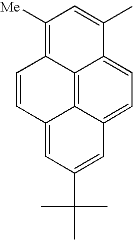 | directbond | 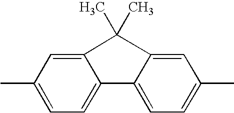 | 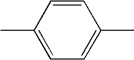 | 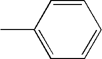 | 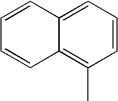 |
| C39 | 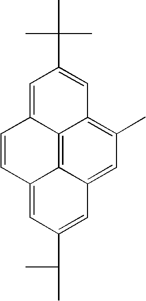 | directbond | 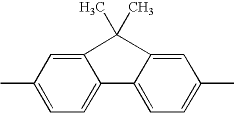 | 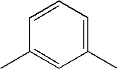 | 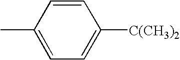 | 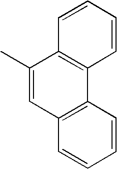 |
| C40 | 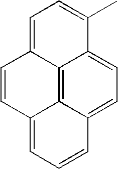 | 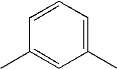 | 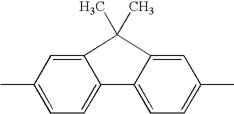 | 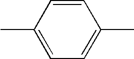 | 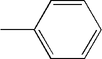 | 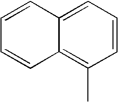 |
| C41 | 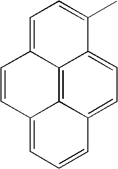 | 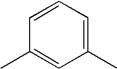 | 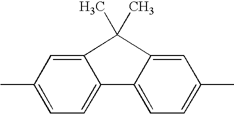 | 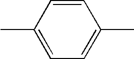 | 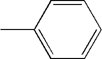 | 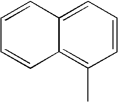 |
| C42 | 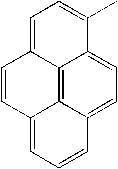 | directbond | 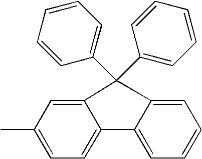 | directbond | 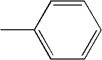 | 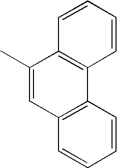 |
| C43 | 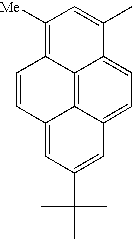 | directbond | 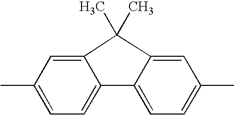 | directbond | 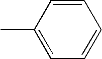 | 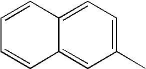 |
| C44 | 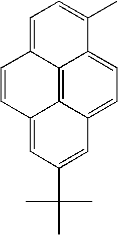 | directbond | 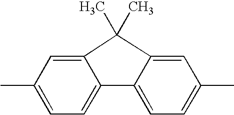 | directbond | 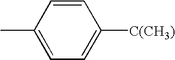 | 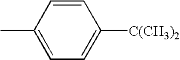 |
| C45 | 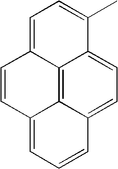 | 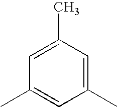 | 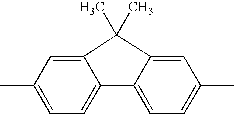 | directbond | 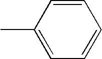 | 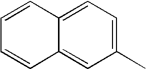 |

TABLE 6

| | | | | | | |
|---|---|---|---|---|---|---|
| C46 | 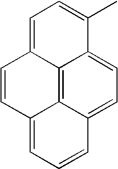 | 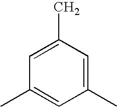 | 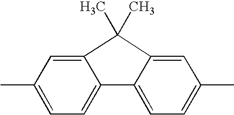 | directbond | 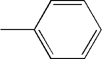 | 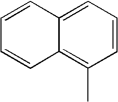 |
| C47 | 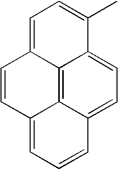 | 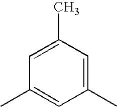 | 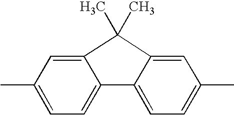 | directbond | 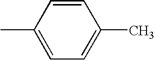 | 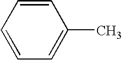 |
| C48 | 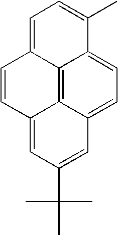 | 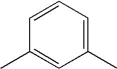 | 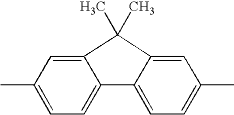 | directbond | 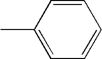 | 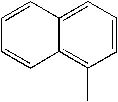 |

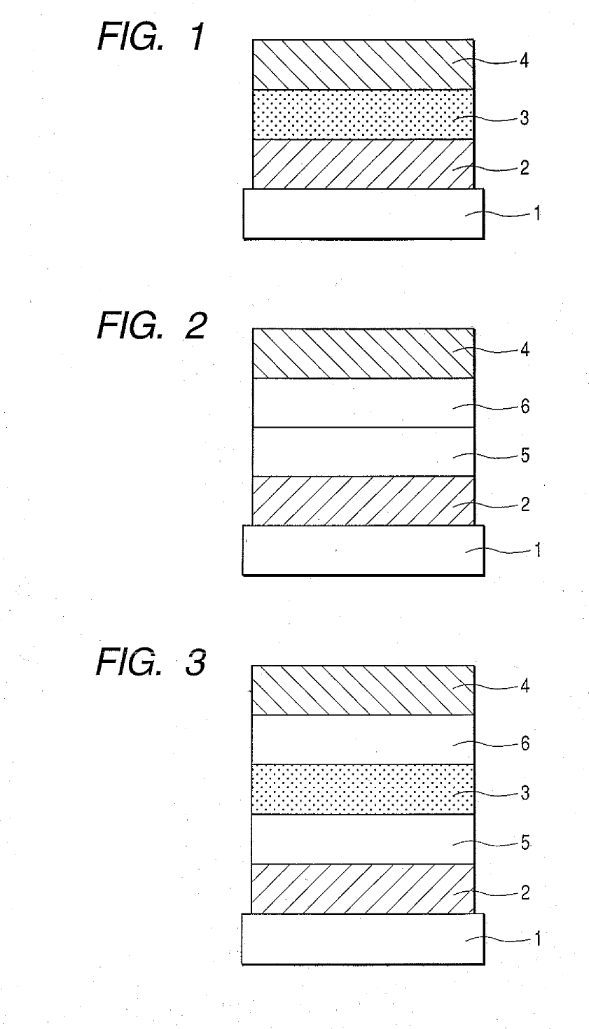

FIGS. 1 to 5 illustrate preferable examples of the organic light-emitting device according to the present invention.

First, the reference numerals in the figures will be explained.

Reference numeral 1 denotes a substrate, reference numeral 2 denotes an anode, reference numeral 3 denotes a light-emitting layer, reference numeral 4 denotes a cathode, reference numeral 5 denotes a hole-transporting layer, reference numeral 6 denotes an electron-transporting layer, reference numeral 7 denotes a hole injection layer, and reference numeral 8 denotes a hole/exciton blocking layer.

FIG. 1 is a cross-sectional view showing an example of the organic light-emitting device according to the present invention. In FIG. 1, the device has a configuration in which an anode 2, a light-emitting layer 3, and a cathode 4 are provided sequentially on a substrate 1. A light-emitting device with this configuration is advantageous when the light-emitting material itself has all of hole transportability, electron transportability, and light-emitting property, or when compounds, respectively, having these characteristics are used in combination.

FIG. 2 is a cross-sectional view showing another example of the organic light-emitting device according to the present invention. In FIG. 2, the device has a configuration such that an anode 2, a hole-transporting layer 5, an electron-transporting layer 6, and a cathode 4 are formed sequentially on a substrate 1. A light-emitting device with this configuration is advantageous when a light-emitting material having either or both of hole transportability and electron transportability is used for the respective layers, in combination with a hole-transporting material having no light-emitting property or an electron-transporting material having no light-emitting property. In addition, in this case, either one of the hole-transporting layer 5 and the electron-transporting layer 6 also serves as the light-emitting layer.

FIG. 3 is a cross-sectional view showing still another example of the organic light-emitting device according to the present invention. In FIG. 3, the device has a configuration in which an anode 2, a hole-transporting layer 5, a light-emitting layer 3, an electron-transporting layer 6, and a cathode 4 are formed sequentially on a substrate 1. With this configuration, the carrier-transporting function and the light-emitting function are separated from each other, so that compounds, respectively, having hole-transporting property, electron-transporting property, and light-emitting property can be used appropriately in combination. Thus, the degree of freedom in selecting materials greatly increases, and various kinds of compounds having different emission wavelengths can be used, whereby a variety of emission wavelengths can be achieved. Furthermore, it also becomes possible to effectively confine carriers or excitons in the light-emitting layer 3 at the middle portion, to thereby increase the emission efficiency.

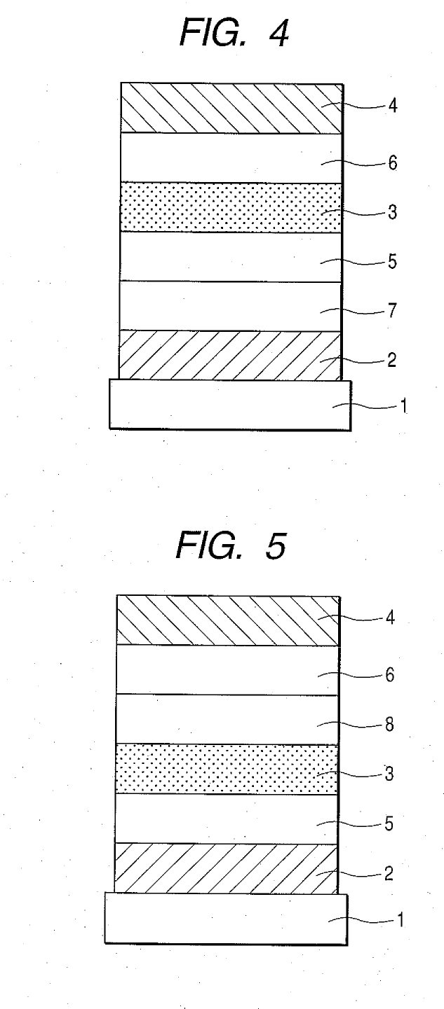

FIG. 4 is a cross-sectional view showing still another example of the organic light-emitting device according to the present invention. In FIG. 4, as compared with FIG. 3, the device is constructed such that a hole injection layer 7 is provided on the anode 2 side, which is effective for improving adhesion between the anode 2 and the hole-transporting layer 5 or improving the hole injection property, thus being effective for reducing the driving voltage.

FIG. 5 is a cross-sectional view showing yet still another example of the organic light-emitting device according to the present invention. In FIG. 5, as compared with FIG. 3, the device is constructed such that a layer (a hole/exciton blocking layer 8) serving to prevent holes or excitons from passing through toward the cathode 4 is provided between the light-emitting layer 3 and the electron-transporting layer 6. Using a compound having an extremely high ionization potential for the hole/exciton blocking layer 8 is effective for improving the emission efficiency.

It is to be noted that FIGS. 1 to 5 merely show very basic device configurations, and that the structure of the organic light-emitting device according to the present invention is not limited thereto. For example, it is possible to adopt various layer configurations, such as one in which an insulating layer is provided at an interface between an electrode and an organic layer, one in which an adhesive layer or an interference layer is provided, or one in which a hole-transporting layer is composed of two layers with different ionization potentials.

The fluorenylene compound according to the present invention can be used in at least any of the configurations illustrated in FIGS. 1 to 5.

Especially, an organic layer using the fluorenylene compound according to the present invention is useful for a light-emitting layer, an electron-transporting layer, or a hole-transporting layer. In addition, such a layer, when formed by vacuum deposition method, solution coating method or the like, is less susceptible to crystallization and is thus excellent in stability over time.

In the present invention, the fluorenylene compound according to the present invention is used as a component for a light-emitting layer in particular. However, such fluorenylene compound can be used in combination with hitherto known low-molecular or polymeric hole-transporting compounds, light-emitting compounds, electron-transporting compounds, or the like.

Examples of such compounds will be now described below.

It is preferable that the hole injecting/transporting material facilitates injection of holes from an anode and has an excellent mobility for transporting the injected holes to a light-emitting layer. Examples of low molecular and high molecular materials having the hole injecting/transporting capability include a triarylamine derivative, a phenylenediamine derivative, a triazole derivative, an oxadiazole derivative, an imidazole derivative, a pyrazoline derivative, a pyrazolone derivative, an oxazole derivative, a fluorenone derivative, a hydrazone derivative, a stilbene derivative, a phthalocyanine derivative, a porphyrin derivative, poly(vinylcarbazole), poly(silylene), poly(thiophen) and other conductive polymers, but is not limited to them.

Other than the fluorenylene compounds according to the present invention, examples of materials mainly relating to light-emitting function which can be used the present invention include, but of course not limited to, fused polycyclic aromatic compounds (e.g. naphthalene derivatives, phenanthrene derivatives, fluorene derivatives, pyrene derivatives, tetracene derivatives, coronene derivatives, chrysene derivatives, perylene derivatives, 9,10-diphenylanthracene derivatives, rubrene and the like), quinacridone derivatives, acridone derivatives, coumarin derivatives, pyran derivatives, nile red, pyrazine derivatives, benzoimidazole derivatives, benzothiazole derivatives, benzoxazole derivatives, stilbene derivatives, organometallic complexes (e.g. organoaluminum complexes such as tris(8-quinolinolate)aluminum and the like, and organoberyllium complexes), and polymer derivatives such as poly(phenylenevinylene) derivatives, poly(fluorene) derivatives, poly(phenylene) derivatives, poly(thienylenevinylene) derivatives, poly(acetylene) derivatives and the like.

The electron injecting/transporting material can be arbitrarily selected from those materials which facilitate injection of electrons from a cathode and have a function of transporting the injected electrons to a light-emitting layer, and is selected in consideration of a balance with the carrier mobility of the hole-transporting material. Examples of the material having electron injecting/transporting capability include, but is not limited to, an oxadiazole derivative, an oxazole derivative, a thiazole derivative, a thiadiazole derivative, a pyrazine derivative, a triazole derivative, a triazine derivative, a perylene derivative, a quinoline derivative, a quinoxaline derivative, a fluorenone derivative, an anthrone derivative, a phenanthroline derivative and an organometallic complex.

In the organic light-emitting device of the present invention, the layer containing the fluorenylene compound of the present invention and the other layers comprising an organic compound can be formed as a thin film generally with vacuum deposition method, ion plating method, sputtering, plasma CVD, or the like. Alternatively, they can be formed as a thin film using a well-known coating method of applying such organic compound dissolved in a suitable solvent, such as spin coating, dipping, casting, LB method, or ink jet method. Particularly, when the film is formed with the coating method, the film can be formed by additionally using a suitable binder resin.

The above described binder resin can be selected from a wide range of binding resins, and includes, for instance, polyvinylcarbazole resin, polycarbonate resin, polyester resin, polyarylate resin, polystyrene resin, ABS resin, polybutadiene resin, polyurethane resin, acrylic resin, methacrylic resin, butyral resin, polyvinylacetal resin, polyamide resin, polyimide resin, polyethylene resin, polyether sulfonic resin, diallylphthalate resin, phenolic resin, epoxy resin, silicone resin, polysulfonic resin and urea resin, but is not limited to them. In addition, the binder resin may be singly used, or be used in combination as a copolymer. Furthermore, an additive such as a well-known plasticizer, antioxidant, and ultraviolet absorber, as needed.

An anode material used preferably has as large a work function as possible, and includes, for instance, an elemental metal such as gold, platinum, silver, copper, nickel, palladium, cobalt, selenium, vanadium and tungsten, an alloy thereof, and a metal oxide such as stannic oxide, zinc oxide, indium oxide, indium tin oxide (ITO) and indium zinc oxide. Further, a conductive polymer such as polyaniline, polypyrrole, polythiophene and polyphenylene sulfide can be employed. These electrode materials can be used singly or in combination. In addition, the anode may be either of a single layer configuration or of a multilayer configuration.

On the other hand, a cathode material used preferably has a low work function, and include, for instance an elemental metal such as lithium, sodium, potassium, calcium, magnesium, aluminum, indium, ruthenium, titanium, manganese, yttrium, silver, lead, tin, and chromium; or an alloy made of a plurality of the above metals, such as lithium-indium, sodium-potassium, magnesium-silver, aluminum-lithium, aluminum-magnesium, and magnesium-indium. A metal oxide such as indium tin oxide (ITO) can be also used. These electrode materials can be used singly or in combination. In addition, the cathode may be either of a single layer configuration or of a multilayer configuration.

A substrate used in the present invention is not particularly limited, but an opaque substrate such as a metal substrate and a ceramic substrate or a transparent substrate such as glass, quartz, and a plastic sheet is used. Further, it is also possible to employ, for a substrate, a color filter film, a fluorescent color conversion filter film and a dielectric reflective film to thereby control the emission color.

Incidentally, after a device has been produced, a protective layer or an encapsulation layer may further be provided, for the purpose of preventing contact with oxygen or moisture. Examples of such a protective layer include a diamond thin film; a film of an inorganic material such as a metal oxide and a metal nitride; a film of a polymer such as a fluororesin, poly-p-xylene, polyethylene, silicone resin, and polystyrene resin; and further a film of a photocurable resin. Further, the produced device may also be covered with glass, a gas-impermeable film and a metal, or be packaged with a suitable encapsulation resin.

The device of the present invention may also be produced in connection to a thin film transistor (TFT) made on a substrate.

Further, as to the direction in which light is extracted out of the device, any one of a bottom emission configuration (configuration in which light is taken out from a substrate side) and a top emission configuration (configuration in which light is taken out from a side opposite to the substrate side) may be adopted as needed.

### EXAMPLES

The present invention will be now described more in detail below with reference to examples, but the present invention is not limited to the examples.

### Example 1

### Production Method for Exemplary Compound No. 1

(1) Synthesis of Intermediate

Under a nitrogen flow, 2 g (5.01 mmol) of the below compound 1 and 2 g (5.01 mmol) of the below compound 3 derived from the below compound 2 were dissolved in a degassed mixed solvent consisting of 80 ml of toluene and 40 ml of ethanol, followed by stirring. The resultant mixture was added with an aqueous solution of sodium carbonate prepared by dissolving 1.1 g of anhydrous sodium carbonate in 15 ml of water. The resultant solution was stirred in an oil bath heated to 50° C., and then added with 289 mg (0.25 mmol) of tetrakis(triphenylphosphine)palladium. Under a nitrogen flow, the solution was heated with stirring for about 4 hours in an oil bath heated to 70° C.

The reaction solution was returned to room temperature, and toluene, ethyl acetate and water were added thereto. The organic layer was separated and dried over magnesium sulfate, and the solvents were evaporated. The residue was purified by silica gel column chromatography (toluene:heptane=1:3) to give 1.1 g of the below intermediate.
Compound 1: 2-bromo-7-iodo-9,9-dimethylfluorene
Compound 2: 7-tert-butyl-3-methyl-1-bromopyrene(synthesized according to “Organic Preparations and Procedures International” (1997), 29, 321-330)
Compound 3: 2-(7-tert-butyl-3-methylpyrene-1-yl)-4,4,5,5-tetramethyl-[1,3,2]dioxaborolane
Intermediate: 1-(7-bromo-9,9-dimethyl-9H-fluorene-2-yl)-7-tert-butyl-3-methylpyrene

(2) Synthesis of Exemplary Compound 1

Under a nitrogen flow, 1 g (1.84 mmol) of the above intermediate and 0.73 g (2.2 mmol) of the below compound 4 were dissolved in a degassed mixed solvent consisting of 60 ml of toluene and 30 ml of ethanol, followed by stirring. The resultant mixture was added with an aqueous sodium carbonate solution prepared by dissolving 0.47 g of anhydrous sodium carbonate in 10 ml of water. The resultant solution was stirred in an oil bath heated to 50° C. Then, the solution was added with 106 mg (0.092 mmol) of tetrakis(triphenylphosphine)palladium and was heated in an oil bath heated to 80° C. with stirring under a nitrogen flow for about 5 hours.

The reaction solution was returned to room temperature, and toluene, ethyl acetate and water were added thereto. The organic layer was separated and dried over magnesium sulfate, and the solvents were evaporated. The residue was purified by silica gel column chromatography (toluene:heptane=1:3) to give 0.74 g of Exemplary Compound 1.
Compound 4: 2-(pyrene-1-yl)-4,4,5,5-tetramethyl-[1,3,2]dioxaborolane

### Example 2

### Production Method for Exemplary Compounds Nos. 14 and 15

Exemplary Compounds 14 and 15 were produced by following the same procedure as in Example 1, with the exception that the below compounds 5 and 6 were used in place of compound 4.
Compound 5: 2-(7-iso-propyl-3-methylpyrene-1-yl)-4,4,5,5-tetramethyl-[1,3,2]dioxaborolane
Compound 6: 2-(7-adamantane-2-yl-3-methylpyrene-1-yl)-4,4,5,5-tetramethyl-[1,3,2]dioxaborolane

### Example 3

### Production Method for Exemplary Compound No. 3

Under a nitrogen flow, 3 g (6.73 mmol) of the below compound 7 and 1.89 g (14.8 mmol) of the below compound 8 were dissolved in a mixed solvent consisting of 100 ml of toluene and 50 ml of ethanol, followed by stirring. The resultant mixture was added with an aqueous sodium carbonate solution prepared by dissolving 1.7 g of anhydrous sodium carbonate in 25 ml of water. The resultant solution was stirred in an oil bath heated to 50° C., and then added with 855 mg (0.74 mmol) of tetrakis(triphenylphosphine)palladium. Under a nitrogen flow, the solution was heated with stirring in an oil bath heated to 80° C. for about 5 hours.

The reaction solution was returned to room temperature, and toluene, ethyl acetate and water were added thereto. The organic layer was separated and dried over magnesium sulfate, and the solvents were evaporated. The residue was purified by silica gel column chromatography (toluene:heptane=1:3) to give 2.5 g of Exemplary Compound 3.
Compound 7: 2,7-bis[2-(4,4,5,5-tetramethyl-[1,3,2]dioxaborolanyl)-9,9-dimethylfluorene
Compound 8: 7-tert-butyl-3-methyl-1-bromopyrene

### Example 4

An organic light-emitting device having a structure shown in FIG. 3 was prepared with a method described below.

A transparent conductive support substrate was prepared which had a film of indium tin oxide (ITO) with a thickness of 120 nm as an anode 2 formed on a glass substrate 1 by a sputtering method. The transparent conductive support substrate was ultrasonically cleaned sequentially with acetone and isopropyl alcohol (IPA), subsequently cleaned with boiled IPA, was dried, was further cleaned with UV/ozone, and was used.

A chloroform solution of a compound represented by the following structural formula, which was a hole-transporting material, was prepared so that the concentration became 0.2 wt. %.

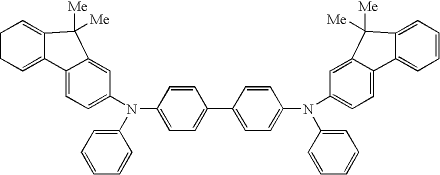

The solution was dropped on the above described ITO electrode and spin-coated at first for ten seconds at a rotation speed of 500 RPM and then for one minute at a rotation speed of 1,000 RPM, to form a film. The substrate was dried in a vacuum oven at 80° C. for ten minutes to completely remove the solvent in the thin film. The formed hole-transporting layer 5 had a thickness of 25 nm.

Subsequently, a light-emitting layer 3 with a thickness of 20 nm was provided on the hole-transporting layer 5 by vapor-depositing the above exemplified compound No. 1. As for the vapor depositing conditions in forming the film, the vacuum degree was 1.0×10⁻⁴ Pa and the film-forming rate was 0.2 to 0.3 nm/sec.

Furthermore, an electron-transporting layer 6 made of bathophenanthroline (BPhen) was formed into a film thickness of 50 nm through a vacuum deposition method. As for the vapor depositing conditions, the vacuum degree was 1.0×10⁻⁴ Pa and the film-forming rate was 0.2 to 0.3 nm/sec.

Then, an organic light-emitting device was prepared by the subsequent steps of: forming a film of a lithium fluoride alloy with a thickness of 0.5 nm on the above described organic layer by a vacuum deposition method; and further providing an aluminum film with a thickness of 150 nm thereon by a vacuum deposition method to make it an electron-injecting electrode (cathode 4). As for the vapor depositing conditions in forming the films, the vacuum degree was 1.0×10⁻⁴ Pa, the film-forming rate for lithium fluoride was 0.05 nm/sec and the film-forming rate for aluminum was 1.0 to 1.2 nm/sec.

The thus obtained organic light-emitting device was covered with a protective glass plate in a dry air atmosphere so that the device was not degraded through adsorbing moisture, and was encapsulated with an acrylic resin adhesive.

When a voltage of 4 V was applied to thus obtained device with the ITO electrode (anode 2) being used as a positive electrode and the aluminum electrode (cathode 4) being used as a negative electrode, emission of a blue light was observed with an emission luminance of 10,500 cd/m².

Furthermore, when a voltage was applied to the device in a nitrogen atmosphere for 100 hours so that the current density was kept at 100 mA/cm², the device emitted light at a luminance of 12,500 cd/m² in an early stage and at about 10,200 cd/m² after the elapse of the 100 hours, which meant that the luminance degradation was small.

### Examples 5 to 7

Devices were prepared by following the same procedure as in Example 4 with the exception that the compounds shown in Table 7 below were used in place of the above exemplified compound No. 1, and were similarly evaluated. The results are shown in Table 7.

TABLE 7

| | Exemplified | Applied | |
|---|---|---|---|
| Example | compound | voltage | Luminance |
| No. | No. | (V) | (cd/m²) |
| 4 | 1 | 4.0 | 10500 |
| 5 | 3 | 4.0 | 12000 |
| 6 | 5 | 4.0 | 9800 |
| 7 | 14 | 4.0 | 11000 |

### Comparative Example 1

A device was prepared by following the same procedure as in Example 4 with the exception that Comparative Compound 1 shown below was used in place of the above exemplified compound No. 1, and were similarly evaluated.

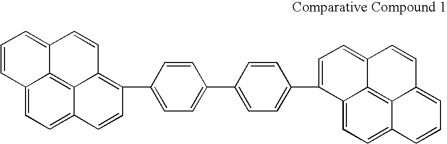

When a voltage of 4 V was applied to the device, emission of a blue light was observed with an emission luminance of 5,500 cd/m². Furthermore, when a voltage was applied to the device in nitrogen atmosphere for 100 hours so that the current density was kept at 100 mA/cm², the device emitted light at a luminance of 6,800 cd/m² in an early stage and of 2,100 cd/m² after elapse of 100 hours, which meant that the luminance degradation was large.

### Examples 8 to 20

Devices were prepared by following the same procedure as in Example 4 with the exception that a light-emitting layer 3 with a thickness of 20 nm was provided by co-depositing the first compound and the second compound shown in Table 8 at the codeposition ratio shown in Table 8; and were similarly evaluated.

The results are shown in Table 8.

Furthermore, when a voltage was applied to the device prepared in Example 11 in nitrogen atmosphere for 100 hours so that the current density was kept at 30 mA/cm², the device emitted light at a luminance of 3,040 cd/m² in an early stage and of 2,070 cd/m² after elapse of 100 hours, which meant that the luminance degradation was small.

TABLE 8

| | | | Codeposition | | |
|---|---|---|---|---|---|
| | 1st | 2nd | Ratio | Applied | |
| Ex. | Comp. | Comp. | (1st Comp.:2nd | Voltage | Luminance |
| No. | No. | No. | Comp.) | (V) | (cd/m²) |
| 8 | AA-12 | 1 | 20:80 | 4.0 | 10800 |
| 9 | AC-1 | 1 | 20:80 | 4.0 | 12100 |
| 10 | AA-20 | 3 | 15:85 | 4.0 | 13100 |
| 11 | AA-33 | 3 | 20:80 | 4.0 | 12600 |
| 12 | BA-1 | 3 | 20:80 | 4.0 | 13800 |
| 13 | C35 | 3 | 15:85 | 4.0 | 12900 |
| 14 | C43 | 3 | 15:85 | 4.0 | 12400 |
| 15 | BA-3 | 5 | 25:80 | 4.0 | 11100 |
| 16 | BB-9 | 5 | 15:85 | 4.0 | 10300 |
| 17 | C34 | 5 | 15:85 | 4.0 | 10500 |
| 18 | BA-6 | 14 | 20:80 | 4.0 | 11800 |
| 19 | BA-11 | 19 | 20:80 | 4.0 | 10700 |
| 20 | BB-14 | 19 | 20:80 | 4.0 | 9400 |

### Comparative Example 2

A device was prepared by following the same procedure as in Example 11 with the exception that Comparative Compound 1 shown above was used as the second compound, and were similarly evaluated.

When a voltage of 4 V was applied to the device, emission of a blue light was observed with an emission luminance of 6,200 cd/m². Furthermore, when a voltage was applied to the device in nitrogen atmosphere for 100 hours so that the current density was kept at 90 mA/cm², the device emitted light at a luminance of 6,500 cd/m² in an early stage and of 1,800 cd/m² after elapse of 100 hours, which meant that the luminance degradation was large.

### Example 21

On a transparent conductive support substrate similar to that prepared in Example 4, a solution of 1.00 g of poly(N-vinylcarbazole) (weight average molecular weight: 63,000) in 80 mL of chloroform was coated in a film thickness of 110 nm by spin coating method (rotation frequency: 2,000 rpm) to form an organic layer (hole-transporting layer).

Then, a solution of 0.050 g of the above exemplified compound No. 8) in 50 mL of toluene was coated in a film thickness of 120 nm by spin coating method (rotation frequency: 2,000 rpm) to form a light-emitting layer.

Next, a cathode was formed and encapsulation was performed in a manner similar to that described in Example 4.

When a voltage of 4 V was applied to the thus obtained device, emission of a blue light was observed with an emission luminance of 2,600 cd/m². Furthermore, when a voltage was applied to the device in nitrogen atmosphere for 100 hours so that the current density was kept at 60 mA/cm², the device emitted light at a luminance of 5,100 cd/m² in an early stage and of 3,900 cd/m² after elapse of 100 hours, which meant that the luminance degradation was small.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2005-366184, filed Dec. 28, 2005, which is hereby incorporated by reference herein in its entirety.

What is claimed is:

1. A fluorenylene compound represented by the general formula (1): 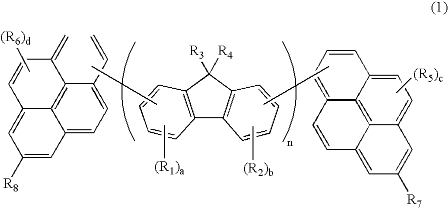
wherein the two pyrenyl groups are each independently bonded at position 1 or 4 thereof to the fluorenylene group;
R₁ and R₂ each represent a substituted or unsubstituted alkyl group, a substituted or unsubstituted aralkyl group, a substituted or unsubstituted aryl group, a substituted or unsubstituted heterocyclic group, a substituted amino group, a cyano group, or a halogen atom, in which R₁'s and R₂'s which are bonded to different fluorene rings may be, respectively, the same or different from each other and R₁ and R₂ which are bonded to the same fluorene ring may be the same or different from each other, and when “a” and “b” are each an integer of 2 or more, R₁'s and R₂'s may be, respectively, the same or different from each other;
R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group, in which R₃'s and R₄'s which are bonded to different fluorene rings may be, respectively, the same or different from each other and R₃ and R₄ which are bonded to the same fluorene ring may be the same or different from each other;
R₅ represents a substituted or unsubstituted alkyl group, a substituted or unsubstituted aralkyl group, a substituted or unsubstituted alkoxy group, a substituted or unsubstituted aryl group, or a substituted or unsubstituted heterocyclic group, and when “c” is an integer of 2 or more, R₅'s may be the same or different from each other;
R₆ represents a substituted or unsubstituted alkyl group having 1 or 2 carbons, a substituted or unsubstituted aralkyl group, a substituted or unsubstituted alkoxy group, a substituted or unsubstituted aryl group, or a substituted or unsubstituted heterocyclic group, and when “d” is an integer of 2 or more, R₆'s may be the same or different from each other;
R₇ and R₈ each independently represent an iso-propyl group, a sec-butyl group, a tert-butyl group, a 1-adamantyl group, a 2-adamantyl group, an iso-amyl group, or a substituted Silyl group;
“a” and “b” each represent an integer from 0 to 3, in which “a's” and “b's” on different fluorene rings may be, respectively, the same or different from each other;
“c” and “d” each represent an integer from 0 to 8, “c”+“d” being an integer from 1 to 16; and
“n” represents an integer from 1 to 10.

2. A fluorenylene compound represented by the general formula (2): 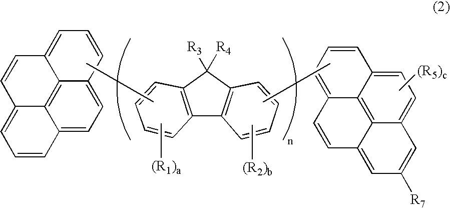
wherein the two pyrenyl groups are each independently bonded at position 1 or 4 thereof to the fluorenylene group;
R₁ and R₂ each represent a substituted or unsubstituted alkyl group, a substituted or unsubstituted aralkyl group, a substituted or unsubstituted aryl group, a substituted or unsubstituted heterocyclic group, a substituted amino group, a cyano group, or a halogen atom, in which R₁'s and R₂'s which are bonded to different fluorene rings may be, respectively, the same or different from each other and R₁ and R₂ which are bonded to the same fluorene ring may be the same or different from each other, and when “a” and “b” are each an integer of 2 or more, R₁'s and R₂'s may be, respectively, the same or different from each other;
R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group, in which R₃'s and R₄'s which are bonded to different fluorene rings may be, respectively, the same or different from each other and R₃ and R₄ which are bonded to the same fluorene ring may be the same or different from each other;
R₅ represents a substituted or unsubstituted alkyl group, a substituted or unsubstituted aralkyl group, a substituted or unsubstituted alkoxy group, a substituted or unsubstituted aryl group, or a substituted or unsubstituted heterocyclic group, and when “c” is an integer of 2 or more, R₅'s may be the same or different from each other;
R₇ represents an iso-propyl group, a sec-butyl group, a tert-butyl group, a 1-adamantyl group, a 2-adamantyl group, an iso-amyl group, or a substituted Silyl group;
“a” and “b” each represent an integer from 0 to 3, in which “a's” and “b's” on different fluorene rings may be, respectively, the same or different from each other;
“c” represents an integer from 1 to 8; and
“n” represents an integer from 1 to 10.

3. The fluorenylene compound according to claim 1, which is represented by the general formula (3):

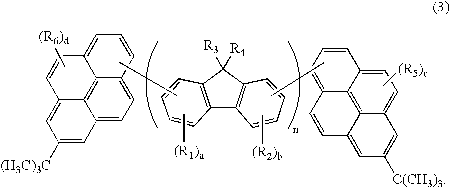

4. The fluorenylene compound according to claim 3, which is represented by the general formula (4):

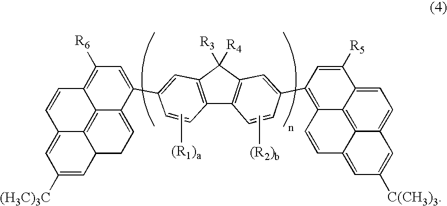

5. The fluorenylene compound according to claim 4, which is represented by the general formula (5): 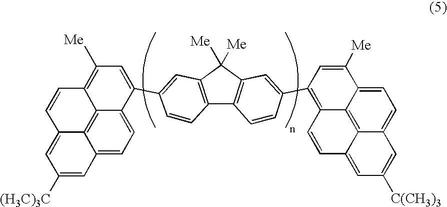
wherein “n” is from 1 to 3.

6. An organic light-emitting device comprising a pair of electrodes including an anode and a cathode at least one of which is transparent or translucent, and at least one layer comprising an organic compound provided between the pair of electrodes, wherein at least one layer of the at least one layer comprising the organic compound comprises at least one of the fluorenylene compounds set forth in claim 1.

7. The organic light-emitting device according to claim 6, wherein the layer comprising at least one of the fluorenylene compounds is a light-emitting layer.

8. The organic light-emitting device according to claim 7, wherein the light-emitting layer comprises an arylamine compound represented by the general formula (6): 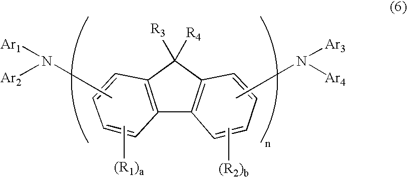
wherein R₁ and R₂ each represent a substituted or unsubstituted alkyl group, a substituted or unsubstituted aralkyl group, a substituted or unsubstituted aryl group, a substituted or unsubstituted heterocyclic group, a substituted amino group, a cyano group, or a halogen atom, in which R₁'s and R₂'s which are bonded to different fluorene rings may be, respectively, the same or different from each other and R₁ and R₂ which are bonded to the same fluorene ring may be the same or different from each other, and when “a” and “b” are each an integer of 2 or more, R₁'s and R₂'s may be, respectively, the same or different from each other;
R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group, in which R₃'s and R₄'s which are bonded to different fluorene rings may be, respectively, the same or different from each other and R₃ and R₄ which are bonded to the same fluorene ring may be the same or different from each other;
Ar₁, Ar₂, Ar₃, and Ar₄ each represent a substituted or unsubstituted aromatic group, a substituted or unsubstituted heterocyclic group, a substituted or unsubstituted fused polycyclic aromatic group, or a substituted or unsubstituted fused heteropolycyclic group, in which Ar₁, Ar₂, Ar₃, and Ar₄ may be the same or different from one another, and Ar₁ and Ar2, and Ar₃ and Ar₄ can be, respectively, joined to form a ring;
“a” and “b” each represent an integer from 0 to 3, in which “a's” and “b's” for different fluorene rings may be, respectively, the same or different from each other; and
n” represents an integer from 1 to 10.

9. The organic light-emitting device according to claim 7, wherein the light-emitting layer comprises an arylamine compound represented by the general formula (7): 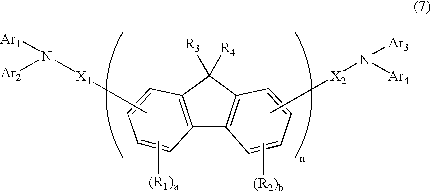
wherein R₁ and R₂ each represent a substituted or unsubstituted alkyl group, a substituted or unsubstituted aralkyl group, a substituted or unsubstituted aryl group, a substituted or unsubstituted heterocyclic group, a substituted amino group, a cyano group, or a halogen atom, in which R₁'s and R₂'s which are bonded to different fluorene rings may be, respectively, the same or different from each other and R₁ and R₂ which are bonded to the same fluorene ring may be the same or different from each other, and when “a” and “b” are each an integer of 2 or more, R₁'s and R₂'s may be, respectively, the same or different from each other;
R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group, in which R₃'s and R₄'s which are bonded to different fluorene rings may be, respectively, the same or different from each other and R₃ and R₄ which are bonded to the same fluorene ring may be the same or different from each other;
Ar₁, Ar₂, Ar₃, and Ar₄ each represent a substituted or unsubstituted aromatic group, a substituted or unsubstituted heterocyclic group, a substituted or unsubstituted fused polycyclic aromatic group, or a substituted or unsubstituted fused heteropolycyclic group, in which Ar₁, Ar₂, Ar₃, and Ar₄ may be the same or different from one another, and Ar₁ and Ar₂, and Ar₃ and Ar₄ can be, respectively, joined to form a ring;
X₁ and X₂ each represent a divalent, substituted or unsubstituted aromatic group or substituted or unsubstituted heterocyclic group, in which X₁ and X₂ may be the same or different from one another;
“a” and “b” each represent an integer from 0 to 3, in which “a's” and “b's” for different fluorene rings may be, respectively, the same or different from each other; and
n” represents an integer from 1 to 10.

10. The organic light-emitting device according to claim 7, wherein the light-emitting layer comprises an arylamine compound represented by the general formula (8): 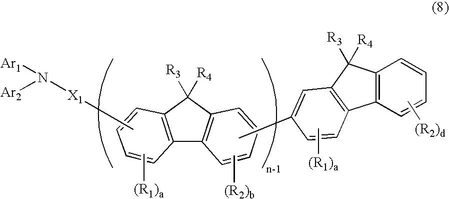
wherein R₁ and R₂ each represent a substituted or unsubstituted alkyl group, a substituted or unsubstituted aralkyl group, a substituted or unsubstituted aryl group, a substituted or unsubstituted heterocyclic group, a substituted amino group, a cyano group, or a halogen atom, in which R₁'s and R₂'s which are bonded to different fluorene rings may be, respectively, the same or different from each other and R₁ and R₂ which are bonded to the same fluorene ring may be the same or different from each other, and when “a” and “b” are each an integer of 2 or more, R₁'s and R₂'s may be, respectively, the same or different from each other;
R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group, in which R₃'s and R₄'s which are bonded to different fluorene rings may be, respectively, the same or different from each other and R₃ and R₄ which are bonded to the same fluorene ring may be the same or different from each other;
Ar₁ and Ar₂ each represent a substituted or unsubstituted aromatic group, a substituted or unsubstituted heterocyclic group, a substituted or unsubstituted fused polycyclic aromatic group, or a substituted or unsubstituted fused heteropolycyclic group, in which Ar₁ and Ar₂ may be the same or different from one another or Ar₁ and Ar₂ can be joined to form a ring; and
X₁ represents a divalent, substituted or unsubstituted aromatic group or substituted or unsubstituted heterocyclic group;
“a” and “b” each represent an integer from 0 to 3, in which “a's” and “b's” for different fluorene rings may be, respectively, the same or different from each other;
d” represents an integer from 0 to 4; and
n” represents an integer from 1 to 10.

11. The organic light-emitting device according to claim 7, wherein the light-emitting layer comprises an arylamine compound represented by the general formula (9): 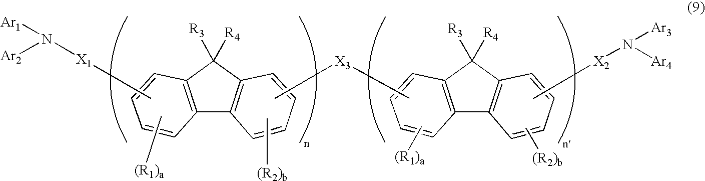
wherein R₁ and R₂ each represent a substituted or unsubstituted alkyl group, a substituted or unsubstituted aralkyl group, a substituted or unsubstituted aryl group, a substituted or unsubstituted heterocyclic group, a substituted amino group, a cyano group, or a halogen atom, in which R₁'s and R₂'s which are bonded to different fluorene rings may be, respectively, the same or different from each other and R₁ and R₂ which are bonded to the same fluorene ring may be the same or different from each other, and when “a” and “b” are each an integer of 2 or more, R₁'s and R₂'s may be, respectively, the same or different from each other;
R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group, in which R₃'s and R₄'s which are bonded to different fluorene rings may be, respectively, the same or different from each other and R₃ and R₄ which are bonded to the same fluorene ring may be the same or different from each other;
Ar₁, Ar₂, Ar₃, and Ar₄ each represent a substituted or unsubstituted aromatic group, a substituted or unsubstituted heterocyclic group, a substituted or unsubstituted fused polycyclic aromatic group, or a substituted or unsubstituted fused heteropolycyclic group, in which Ar₁, Ar₂, Ar₃, and Ar₄ may be the same or different from one another, and Ar₁ and Ar₂, and Ar₃ and Ar₄ can be, respectively, joined to form a ring;
X₁ and X₂ each represent a directly-bonded, divalent, substituted or unsubstituted aromatic group or substituted or unsubstituted heterocyclic group, which can be a group bonded through a linking group, in which X₁ and X₂ may be the same or different from one another;
X₃ is a group selected from the group consisting of a substituted or unsubstituted alkylene group, alkenylene group, alkynylene group, aralkylene group, a divalent, substituted or unsubstituted aromatic group and substituted or unsubstituted heterocyclic group, and can be a group bonded through a linking group;
“a” and “b” each represent an integer from 0 to 3, in which “a's” and “b's” for different fluorene rings may be, respectively, the same or different from each other; and
n” and “n′” each represent an integer from 1 to 10.

12. The organic light-emitting device according to claim 7, wherein the light-emitting layer comprises an arylamine compound represented by the general formula (10): 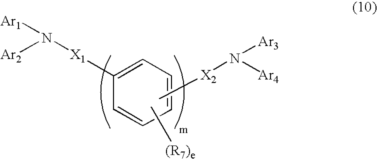
wherein R₇ represents a substituted or unsubstituted alkyl group, a substituted or unsubstituted aralkyl group, a substituted or unsubstituted aryl group, a substituted or unsubstituted heterocyclic group, a cyano group, or a halogen atom, in which R₇'s bonded to different phenylene groups may be the same or different from each other, and when “e” is an integer of 2 or more, R₇'s bonded to the same phenylene group may be the same or different from each other;
Ar₁, Ar₂, Ar3, and Ar₄ each represent a substituted or unsubstituted aromatic group, a substituted or unsubstituted heterocyclic group, a substituted or unsubstituted fused polycyclic aromatic group, or a substituted or unsubstituted fused heteropolycyclic group, in which Ar₁, Ar₂, Ar₃, and Ar₄ may be the same or different from one another, and Ar₁ and Ar₂, and Ar₃ and Ar₄ can be, respectively, joined to form a ring;
X₁ and X₂ each represent a direct bond, or a divalent, substituted or unsubstituted aromatic group or substituted or unsubstituted heterocyclic group, which can be a group bonded through a linking group, in which X₁ and X₂ may be the same or different from one another;
“e” represents an integer from 0 to 4, in which “e's” for different phenylene groups may be the same or different from each other; and
“m” represents an integer from 1 to 10.

13. The organic light-emitting device according to claim 7, wherein the light-emitting layer comprises an arylamine compound represented by the general formula (11): 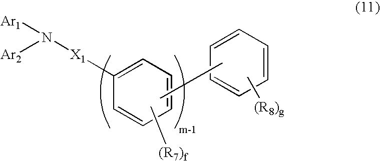
R₇ represents a substituted or unsubstituted alkyl group, a substituted or unsubstituted aralkyl group, a substituted or unsubstituted aryl group, a substituted or unsubstituted heterocyclic group, a cyano group, or a halogen atom, in which R₇'s bonded to different phenylene groups may be the same or different from each other, and when “f” is an integer of 2 or more, R₇'s bonded to the same phenylene group may be the same or different from each other;
R₈ represents a substituted or unsubstituted alkyl group, a substituted or unsubstituted aralkyl group, a substituted or unsubstituted aryl group, a substituted or unsubstituted heterocyclic group, a cyano group, or a halogen atom, and when “g” is an integer of 2 or more, R₈'s may be the same or different from each other;
Ar₁ and Ar₂ each represent a substituted or unsubstituted aromatic group, a substituted or unsubstituted heterocyclic group, a substituted or unsubstituted fused polycyclic aromatic group, or a substituted or unsubstituted fused heteropolycyclic group, in which Ar₁ and Ar₂ may be the same or different from one another or Ar₁ and Ar₂ can be joined to form a ring;
X₁ represents a direct bond, or a divalent, substituted or unsubstituted aromatic group or substituted or unsubstituted heterocyclic group, which can be a group bonded through a linking group;
“f” represents an integer from 0 to 4, in which “f's” for different phenylene groups may be the same or different from each other;
“g” represents an integer from 0 to 5; and
“m” represents an integer from 1 to 10.

14. The organic light-emitting device according to claim 7, wherein the light-emitting layer comprises an acetylene compound represented by the general formula (12): 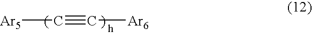
wherein Ar₅ and Ar₆ each represent a substituted or unsubstituted aromatic group, a substituted or unsubstituted heterocyclic group, a substituted or unsubstituted fused polycyclic aromatic group, or a substituted or unsubstituted fused heteropolycyclic group, in which Ar₅ and Ar₆ may be the same or different from one another; and
“h” represents an integer from 1 to 5.

15. The organic light-emitting device according to claim 7, wherein the light-emitting layer comprises an arylamine compound represented by the general formula (13): 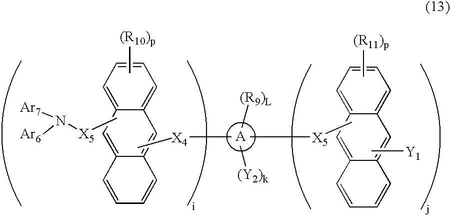
wherein A represents a molecular unit comprising an aromatic ring, a fused polycyclic ring or a heterocyclic ring;
Ar₇ and Ar₈ each represent a group selected from the group consisting of substituted or unsubstituted alkyl group, aralkyl group, aryl group, and heterocyclic group, in which Ar₇ and Ar₈ may be the same or different from each other, or Ar₇ and Ar₈ can be joined to form a ring, and Ar₇'s and Ar₈'s for different anthryl derivative groups may be, respectively, the same or different from each other;
X₄ and X₅ are each a group selected from the group consisting of a direct bond, substituted or unsubstituted alkylene group, alkenylene group, alkynylene group, aralkylene group, and arylene group and a divalent heterocyclic group, which can be a group bonded through a linking group, and may be the same or different from one another, and X₄'s and X₅'s for different anthryl derivative groups may be, respectively, the same or different from each other;
X₆ represents a direct bond, a divalent, substituted or unsubstituted aromatic group or substituted or unsubstituted heterocyclic group, which can be a group bonded through a linking group, and X₆'s for different anthryl derivative groups may be the same or different from each other;
Y₁ is a group selected from the group consisting of a hydrogen atom, a halogen atom, substituted or unsubstituted alkyl group, alkenyl group, alkynyl group, aralkyl group, alkoxy group, sulfide group, aryl group, heterocyclic group, and boranyl group and a substituted silyl group, which may be a group bonded through a linking group, and Y1's for different anthryl derivative groups may be the same or different from each other;
Y₂ is a group selected from the group consisting of substituted or unsubstituted aryl group and heterocyclic group, which may be a group bonded through a linking group, and when “k” is an integer of 2 or more, Y₂'s may be the same or different from each other;
R₉ is a group selected from the group consisting of a hydrogen atom, a halogen atom, substituted or unsubstituted alkyl group and alkoxy group, and when “L” is an integer of 2 or more, R₉'s may be the same or different from each other;
R₁₀ and R₁₁ are each a group selected from the group consisting of a halogen atom, substituted or unsubstituted alkyl group, aryl group, alkoxy group and amino group, and may be the same or different from each other, when “p” and “q” are each an integer of 2 or more, R₁₀'s and R₁₁'s may be, respectively, the same or different from each other, and R₁₀'s and R₁₁'s bonded to different anthracene rings may be, respectively, the same or different from each other;
“i” is an integer from 0 to 6, and the relationship of j+k+L=6−i is satisfied with the proviso that the sum of i+j is an integer of 2 or more, and when “i” is zero at least one of Y₁'s on the anthryl groups contains a substituent other than a hydrogen atom and a halogen atom; and
“p” is an integer from 0 to 8.

16. The organic light-emitting device according to claim 7, wherein the light-emitting layer comprises an arylamine compound represented by the general formula (14): 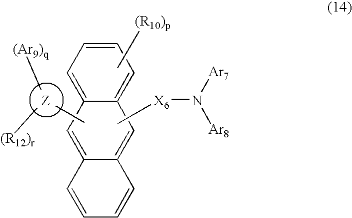
wherein “Z” is a group selected from the group consisting of a direct bond, substituted or unsubstituted alkylene group, alkenylene group, alkynylene group, aralkylene group, and arylene group, and a divalent heterocyclic group, which can be a group bonded through a linking group;
Ar₇ and Ar₈ each represent a group selected from the group consisting of substituted or unsubstituted alkyl group, aralkyl group, aryl group, and heterocyclic group, in which Ar₇ and Ar₈ may be the same or different from each other, or Ar₇ and Ar₈ can be joined to form a ring;
Ar₉ represents a substituent comprising an aromatic fused polycyclic unit selected from the group consisting of substituted or unsubstituted naphthalene, phenanthrene, acenaphthylene, acephenanthrylene, aceanthrylene, triphenylene, chrysene, benzo[c]phenanthrene, naphthacene, dibenzo[a,c]anthracene, dibenzo[a,h]anthracene, dibenzo[b,def]chrysene, pyrene, picene, perylene, and pentacene, and a substituted or unsubstituted heterocyclic group, and when “q” is an integer of 2 or more, Ar₉'s may be the same or different from each other;
X₆ represents a direct bond, a divalent, substituted or unsubstituted aromatic group or substituted or unsubstituted heterocyclic group, which can be a group bonded through a linking group, and X₆'s on different anthryl derivative groups may be the same or different from each other;
R₁₀ is a group selected from the group consisting of a halogen atom, substituted or unsubstituted alkyl group, aryl group, alkoxy group, and amino group, and when “p” is an integer of 2 or more, R₁₀'s may be the same or different from each other;
R₁₂ is a group selected from the group consisting of a halogen atom, substituted or unsubstituted alkyl group, aryl group excluding anthryl group, alkoxy group, and amino group, in which when “r” is an integer of 2 or more, R₁₂'s may be the same or different from each other;
“p” represents an integer from 0 to 8;
“q” represents an integer from 1 to 3; and
“r” represents an integer from 0 to 4.

17. The organic light-emitting device according to claim 7, wherein the light-emitting layer comprises an arylamine compound represented by the general formula (15): 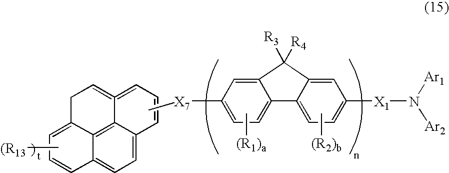
wherein R₁ and R₂ each represent a substituted or unsubstituted alkyl group, a substituted or unsubstituted aralkyl group, a substituted or unsubstituted aryl group, a substituted or unsubstituted heterocyclic group, a substituted amino group, a cyano group, or a halogen atom, in which R₁'s and R₂'s which are bonded to different fluorene rings may be, respectively, the same or different from each other and R₁ and R₂ which are bonded to the same fluorene ring may be the same or different from each other, and when “a” and “b” are each an integer of 2 or more, R₁'s and R₂'s may be, respectively, the same or different from each other;
R₃ and R₄ each represent a hydrogen atom, a substituted or unsubstituted alkyl group, a substituted or unsubstituted aralkyl group, a substituted or unsubstituted aryl group, or a substituted or unsubstituted heterocyclic group, in which R₃'s and R₄'s which are bonded to different fluorene rings may be, respectively, the same or different from each other and R₃ and R₄ which are bonded to the same fluorene ring may be the same or different from each other;
R₁₃ is a group selected from the group consisting of a halogen atom, substituted or unsubstituted alkyl group, aryl group, alkoxy group, and amino group, and when “t” is an integer of 2 or more, R₁₃'s may be the same or different from each other;
Ar₁ and Ar₂ each represent a substituted or unsubstituted aromatic group, a substituted or unsubstituted heterocyclic group, a substituted or unsubstituted fused polycyclic aromatic group, or a substituted or unsubstituted fused heteropolycyclic group, in which Ar₁ and Ar₂ may be the same or different from one another or Ar₁ and Ar₂ can be joined to form a ring;
X₁ represents a direct bond, a divalent, substituted or unsubstituted aromatic group or substituted or unsubstituted heterocyclic group, which can be a group bonded through a linking group;
X₇ is a group selected from the group consisting of substituted or unsubstituted alkylene group, alkenylene group, alkynylene group, aralkylene group, or a divalent, substituted or unsubstituted aromatic group or substituted or unsubstituted heterocyclic group, which may be a group bonded through a linking group;
“a” and “b” each represent an integer from 0 to 3, in which “a's” and “b's” which for different fluorene rings may be, respectively, the same or different from each other;
“t” represents an integer from 0 to 9; and
“n” represents an integer from 1 to 10.
